(19) United States
(12) Patent Application Publication
(10) Pub. No.: US 2025/0079005 A1
(43) Pub. Date: Mar. 6, 2025

(54) ECCDNA REMNANTS AS A CANCER BIOMARKER

(71) Applicant: Centre for Novostics, Shatin (HK)

(72) Inventors: Yuk-Ming Dennis Lo, Homantin (CN); Kwan Chee Chan, Jordan (CN); Peiyong Jiang, Pak Shek Kok (CN); Tsz Kwan Sin, Sham Shui Po (CN); Jiaen Deng, Shatin (CN)

(21) Appl. No.: 18/819,469

(22) Filed: Aug. 29, 2024

Related U.S. Application Data
(60) Provisional application No. 63/535,236, filed on Aug. 29, 2023.

(51) Int. Cl. G16H 50/20; G16B 20/50; G16B 30/10

(52) U.S. Cl. CPC G16H 50/20; G16B 20/50; G16B 30/10

(57) ABSTRACT

Methods and systems are described herein that include using sequence reads of linear DNA molecules naturally present in a biological sample to classify a set of the linear DNA molecules that are eccDNA remnants, i.e., linear DNA molecules resulting from in vivo opening of eccDNA molecules. In various embodiments, characteristics of the classified eccDNA remnants can be analyzed to determine a property of the biological sample or of the subject from whom the biological sample was obtained. Examples of properties that can be determined include a classification of a pathology, e.g., a level of a cancer, or an inferred age of the subject.

### CROSS-REFERENCES TO RELATED APPLICATIONS

The present application claims priority from and is a nonprovisional application of U.S. Provisional Application No. 63/535,236, entitled “eccDNA Remnants as a Cancer Biomarker” filed Aug. 29, 2023, the entire contents of which are herein incorporated by reference for all purposes.

### BACKGROUND

Extrachromosomal DNA (ecDNA) is covalently circularized DNA found outside of the chromosomes. Many studies of these DNA molecules focus on ecDNA shorter than 1 kb, sometimes referred to as extrachromosomal circular DNA (eccDNA) (Yi E, Chamorro González R, Henssen AG & Verhaak RGW, Nat Rev Genet. 23, (2022): 760). Sin et al. found that eccDNA can be present in the maternal plasma DNA of pregnant women and has a size distribution with two major peak clusters at 202 bp and 338 bp, which are remarkably different from the 166-bp peak seen with linear cfDNA molecules (Sin STK et al., Proc. Natl. Acad. Sci. USA 117, (2020): 1658). The fetal-derived eccDNA exhibits relatively shorter sizes and lower methylation levels than eccDNA originating from the mother (Sin STK et al., Proc. Natl. Acad. Sci. USA 117, (2020): 1658; Sin STK et al., Clin. Chem. 67, (2021): 788), suggesting that the generation of eccDNA molecules might be related to their tissues of origin. Recent analyses of tumor tissues indicate that there are also ecDNA with much larger sizes, ranging from 50 kb to 5 Mb, and that these contribute to oncogenic remodeling through chimeric circularization and reintegration of circular DNA into the linear genome (Koche RP et al., Nat. Genet. 52, (2020): 29).

Some methods of eccDNA identification are based on the removal of background linear DNA molecules via exonucleases, followed by the cleavage of the intact circular DNA molecules via restriction enzymes (e.g., MspI) or transposases to facilitate sequencing (Sin STK et al., Proc. Natl. Acad. Sci. USA 117, (2020): 1658). Other methods use rolling circular amplification (RCA) of intact circular DNA molecules remaining after removal of linear DNA with exonucleases, where this amplification is then followed by sonication, sequencing adaptor ligation, and sequencing (Shibata Y et al., Science 336, (2012): 82). Notably, each of these existing procedures includes in vitro steps to enrich eccDNA (e.g., through removal of linear DNA or RCA), as all data generated by random whole genome sequencing without such enrichment steps were presumed to originate from DNA molecules that were always linear.

### BRIEF SUMMARY

Various embodiments are provided for identifying linear DNA molecules that are the products of in vivo cleavage of a circular DNA molecule, e.g., an eccDNA molecule. The identification of these particular linear DNA molecules, termed eccDNA remnants, can involve classifying linear DNA in a sample using sequence information, e.g., sequence reads. Notably, the classifying of the eccDNA remnants of the sample can use sequence information from, e.g., random whole genome sequencing. Accordingly, in vitro operations to enrich and/or break the circular DNA of the sample prior to the sequencing are not required. The sample can be a biological sample, such as a plasma sample obtained from a subject, and can include cellular and/or cell-free DNA.

One example purpose of the classifying of eccDNA remnants in a biological sample from a subject is the determining of a property of the biological sample or of the subject. An exemplary property that can be determined is the classification of a pathology, such as cancer. Another exemplary property is a fractional concentration of clinically relevant DNA as inferred from classified eccDNA remnants. The determining of the property of the biological sample or subject can involve analyzing the classified eccDNA remnants to determine a count of the eccDNA remnants. Alternatively or additionally, the determining of the property can involve determining a size distribution of the eccDNA remnants and/or the eccDNA molecules from which the eccDNA remnants originated. In some examples, the determining of the property can additionally or alternatively include determining a normalized genomic coverage of the eccDNA remnants, determining a frequency of nucleotide motif patterns associated with the eccDNA remnants and/or determining methylation densities of the eccDNA remnants.

In some embodiments, the present disclosure relates to a method for analyzing a biological sample from a subject, where the biological sample includes a plurality of cell-free linear DNA molecules that each independently have a 5′ end sequence and a 3′ end sequence. The method includes, for each of the plurality of cell-free linear DNA molecules, receiving one or more sequence reads having at least the 5′ end sequence and the 3′ end sequence to obtain a 5′ end sequence read and a 3′ end sequence read; mapping the 5′ end sequence and the 3′ end sequence to a reference genome; and based on the mapping, classifying whether the cell-free linear DNA molecule was cleaved in vivo from a circular DNA molecule, thereby identifying a set of eccDNA remnants. The method further includes analyzing the set of eccDNA remnants to determine a property of the biological sample or subject.

These and other embodiments of the disclosure are described in detail below. For example, other embodiments are directed to systems, devices, and computer readable media associated with methods described herein.

Reference to the remaining portions of the specification, including the drawings and claims, will realize other features and advantages of the present disclosure. Further features and advantages of the present disclosure, as well as the structure and operation of various embodiments of the present disclosure, are described in detail below with respect to the accompanying drawings. In the drawings, like reference numbers can indicate identical or functionally similar elements.

### BRIEF DESCRIPTION OF THE DRAWINGS

FIG. 1 presents a schematic illustration of eccDNA remnants, i.e., linear DNA molecules formed by in vivo breakage, e.g., cleavage, of eccDNA molecules.

FIG. 2 illustrates examples of in vivo formation of an eccDNA molecule from genomic DNA, in vivo formation of an eccDNA remnant from the eccDNA molecule, and a workflow for analyzing and classifying the eccDNA remnant by mapping received sequence reads.

FIG. 3 presents schematic illustrations of exemplary mapped sequence reads indicating that a linear DNA molecule includes a deletion, insertion, inversion, or duplication, rather than that the linear DNA molecule is an eccDNA remnant.

FIG. 4 presents a flowchart of a method for classifying whether a cell-free linear DNA molecule is an eccDNA remnant based on mapping of sequence reads of the cell-free linear DNA molecule.

FIG. 5 presents a flowchart of a method for determining a property of a biological sample or of a subject from whom the biological sample was obtained, the determining based on an analysis of linear DNA molecules classified as eccDNA remnants by mapping sequence reads of the linear DNA molecules.

FIG. 6 presents a table of data related to sequence reads from hepatitis B virus (HBV) carriers and hepatocellular carcinoma (HCC) patients, and related to eccDNA remnants identified based on the mapping of the sequence reads.

FIG. 7 presents a graph plotting a comparison between normalized counts of eccDNA remnants detected in HBV carrier biological samples, and normalized counts of eccDNA remnants detected in HCC patient biological samples.

FIG. 8A presents a graph plotting a size-frequency distribution for small eccDNA remnants detected using methods provided herein.

FIG. 8B presents a graph plotting a size-frequency distribution for small eccDNA molecules as measured using paired-end sequencing.

FIG. 9 presents a graph plotting a comparison between percentages of eccDNA remnants detected in HBV carrier biological samples and having a size larger than 1 kb, and percentages of eccDNA remnants detected in HCC patient biological samples and having a size larger than 1 kb.

FIG. 10 presents a graph plotting size-frequency distributions of eccDNA remnants detected in HBV carrier biological samples and HCC patient biological samples.

FIG. 11 presents a table of data related to sequence reads from non-NPC subjects and NPC patients, and related to eccDNA remnants identified based on the mapping of the sequence reads.

FIG. 12 presents a graph plotting a comparison between percentages of eccDNA remnants detected in non-NPC subject biological samples and having a size larger than 1 kb, and percentages of eccDNA remnants detected in NPC patient biological samples and having a size larger than 1 kb.

FIG. 13 presents a graph plotting the genomic distribution of eccDNA remnants detected in HBV carrier samples and HCC patient samples.

FIG. 14 presents an illustration of segments I, II, III, and IV of a nucleotide motif pattern associated with an eccDNA remnants have the designated Start position and End position in the reference genome, where segments I and II are separated by adjoining spacer region S1, and segments III and IV are separated by adjoining spacer region S2.

FIG. 15 presents tables listing frequency data for trinucleotide motif 4-segment patterns associated with eccDNA fragments from HBV carriers and HCC patients.

FIG. 16 presents tables listing frequency data for dinucleotide motif 4-segment patterns associated with eccDNA fragments from HBV carriers and HCC patients.

FIG. 17 presents tables listing frequency data for tetranucleotide motif 4-segment patterns associated with eccDNA fragments from HBV carriers and HCC patients.

FIG. 18 presents tables listing frequency data for dinucleotide motif 4-segment patterns associated with eccDNA fragments from HBV carriers and HCC patients, where the segments of the patterns are located at different genomic positions than those of FIG. 16.

FIG. 19 presents tables listing frequency data for trinucleotide motif 3-segment patterns associated with eccDNA fragments from HBV carriers and HCC patients.

FIG. 20 presents tables listing frequency data for tetranucleotide motif 3-segment patterns associated with eccDNA fragments from HBV carriers and HCC patients.

FIG. 21 presents tables listing frequency data for trinucleotide motif 2-segment patterns associated with eccDNA fragments from HBV carriers and HCC patients.

FIG. 22 presents tables listing frequency data for tetranucleotide motif 2-segment patterns associated with eccDNA fragments from HBV carriers and HCC patients.

FIG. 23 presents tables listing frequency data for dinucleotide motif 2-segment patterns associated with eccDNA fragments from HBV carriers and HCC patients.

FIG. 24 presents tables listing frequency data for trinucleotide motif 1-segment patterns associated with eccDNA fragments from HBV carriers and HCC patients.

FIG. 25 presents tables listing frequency data for tetranucleotide motif 1-segment patterns associated with eccDNA fragments from HBV carriers and HCC patients.

FIG. 26 presents tables listing frequency data for dinucleotide, trinucleotide, and tetranucleotide motif 1-segment patterns located at various genomic positions relative to the start position of eccDNA fragments from HBV carriers and HCC patients.

FIG. 27 presents tables listing frequency data for dinucleotide, trinucleotide, and tetranucleotide motif 1-segment patterns located at various genomic positions relative to the end position of eccDNA fragments from HBV carriers and HCC patients.

FIG. 28 presents a graph plotting a comparison between methylation density percentages for eccDNA remnants of various size ranges detected in HBV carrier biological samples and HCC patient biological samples.

FIG. 29 presents graphs plotting area under the curve (AUC) values for differentiating HBV carrier biological samples from HCC patient biological samples using eccDNA remnants having sizes either below (upper graph) or above (lower graph) various cutoff sizes.

FIG. 30 presents a graph plotting a comparison between methylation density percentages for eccDNA remnants having sizes greater than 1 kb that were detected in HBV carrier biological samples and HCC patient biological samples.

FIG. 31 presents a graph plotting a comparison between methylation density percentages for linear DNA and eccDNA remnants detected in biological samples.

FIG. 32 presents a flowchart of a method for determining a cancer level of a subject, the determining based on an analysis of linear DNA molecules classified as eccDNA remnants by mapping sequence reads of linear DNA molecules in a biological sample from the subject.

FIG. 33 presents a graph showing a correlation between the percentage of eccDNA remnants longer than 1 kb in a biological sample, and the fraction of tumor DNA in the biological sample.

FIG. 34 presents a block diagram of an exemplary measurement system in accordance with a provided embodiment.

FIG. 35 presents a block diagram of an exemplary computer system in accordance with a provided embodiment.

FIG. 36 presents a flowchart of a method of estimating a fractional concentration of clinically-relevant DNA in a biological sample.

FIG. 37 presents a flowchart of a method of determining calibration data points from measurements made from calibration samples.

### TERMS

The term “biological sample” refers to any sample that is taken from a “subject” (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia), and that contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 3,000 g×10 minutes, obtaining the fluid part of the centrifuged sample, and re-centrifuging at for example, 30,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The abbreviation “eccDNA” refers to extrachromosomal circular DNA, which is a covalently closed circular DNA molecule that includes DNA originating from a genome. An eccDNA molecule can have any size.

An “eccDNA remnant” is a linear molecule that is the product of in vivo cleavage of an eccDNA molecule.

The terms “junction,” “junction locus,” and “junction site” refer to a location in a nucleic acid molecule at which two sequences having separated locations (i.e., coordinates) in a reference (e.g., a reference genome) are adjacent to one another. Thus, despite not being adjacent to one another in the reference (e.g., the reference genome) theses sequences are adjacent to one another at the junction of the nucleic acid molecule. As one example, a junction can be created when a segment of genomic DNA is circularized, thereby joining the ends of this segment to form a circular DNA molecule, e.g., an eccDNA molecule.

The term “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000 or 50,000 or 100,000 or 500,000 or 1,000,000 or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

A sequence read can include an “end sequence” or “ending sequence” associated with an end of a DNA molecule, e.g., the 5′ end of the DNA molecule or the 3′ end of the DNA molecule. The end sequence can correspond to the outermost N bases of the molecule, e.g., 1-30 bases at an end of the DNA molecule. If a sequence read corresponds to an entire DNA molecule, then the sequence read can include two ending sequences. When paired-end sequencing provides two sequence reads that correspond to the ends of the DNA molecule, each sequence read can include one ending sequence. A sequence read including the 5′ ending sequence of a DNA molecule is referred to herein as a “5′ end sequence read.” A sequence read including the 3′ ending sequence of a DNA molecule is referred to herein as a “3′ end sequence read.” A sequence read corresponding to an entire DNA molecule is both a “5′ end sequence read” and a “3′ end sequence read.”

The term “mapping” or “alignment” refers to a process which relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

The terms “genome coverage” and “genomic coverage” generally relate to a measure of the regions of a genome (e.g., a reference genome) represented by a nucleic acid molecule (e.g., a DNA molecule), a population of nucleic acid molecules, or the sequences of such molecules. A “normalized genomic coverage” is a value indicating the frequency with which members of a population of nucleic acid molecules or sequences map to particular genomic regions, where the value is normalized with respect to the frequency with which those particular genomic regions occur in the genome. One method for calculating a normalized genomic coverage for a population of nucleic acid molecules or sequences involves determining the percentage of the population that maps to particular regions of the genome (e.g., and then dividing this percentage by the percentage of the total genome that is within those particular regions.

The abbreviation “bp” refers to base pairs. In some instances, “bp” may be used to denote a length of a DNA fragment, even though the DNA fragment may be single stranded and does not include a base pair. In the context of single-stranded DNA, “bp” may be interpreted as providing the length in nucleotides.

The terms “size profile” and “size distribution” generally relate to the sizes of DNA fragments in a biological sample. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range.

The term “nucleotide motif” refers to an arrangement of two or more nucleotides (e.g., two or more adjacent nucleotides) that is overrepresented within a set of nucleotide sequences. A nucleotide motif may or may not be located at a conserved position with the set of nucleotide sequences.

“DNA methylation” in mammalian genomes typically refers to the addition of a methyl group to the 5′ carbon of cytosine residues (i.e., 5-methylcytosines) among CpG dinucleotides.

DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported.

The “methylation index” for each genomic site (e.g., a CpG site) can refer to the proportion of DNA fragments (e.g., as determined from sequence reads or probes) showing methylation at the site over the total number of reads covering that site. A “methylation status” can refer to whether a particular site is methylated at a particular site of a DNA fragment or whether a particular site in a genome has a particular differential methylation status, e.g., hypermethylation or hypomethylation. A “read” can include information (e.g., methylation status at a site) obtained from a DNA fragment. A read can be obtained using reagents (e.g., primers or probes) that preferentially hybridize to DNA fragments of a particular methylation status. Typically, such reagents are applied after treatment with a process that differentially modifies or differentially recognizes DNA molecules depending on their methylation status, e.g., bisulfite conversion, or methylation-sensitive restriction enzyme, or methylation binding proteins, or anti-methylcytosine antibodies, or single molecule sequencing techniques that recognize methylcytosines and hydroxymethylcytosines.

The “methylation density” of a region or a set of sites can refer to the number of reads at site(s) within the region (also referred to as a bin) or the set of sites showing methylation divided by the total number of reads covering the site(s) in the region or the set of sites. A region can include one or more sites of interest, including at least 1, 2, 3, 4, 5, 10, 20, 50, 100, 200, 500, and 1,000 sites. The site(s) may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100 kb region. This analysis can also be performed for other bin sizes, e.g., 500 bp, 5 kb, 10 kb, 50 kb or 1 Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer to the number of cytosine sites, “C's”, that are shown to be methylated (for example unconverted after bisulfite conversion) over the total number of analyzed cytosine residues, i.e., including cytosines outside of the CpG context, in the region. The methylation index, methylation density and proportion of methylated cytosines are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci USA 2013; 110: 18910-18915) and by the Pacific Biosciences single molecule real time analysis (Tse et al. Proc Natl Acad Sci USA 2021; 118: e2019768118).

A “methylation level” is an example of a relative abundance, e.g., between methylated DNA molecules (e.g., at one or more particular sites) and other DNA molecules (e.g., all other DNA molecules or just unmethylated DNA molecules at the one or more particular sites). The amount of other DNA molecules can act as a normalization factor. As another example, an intensity of methylated DNA molecules (e.g., fluorescent or electrical intensity) relative to intensity of all or unmethylated DNA molecules at one or more sites can be determined. The relative abundance can also include an intensity per volume. A methylation level can be determined using a methylation-aware assay such as methylation-aware sequencing or PCR. Example methylation-aware sequencing can include bisulfite sequencing or single molecule techniques, e.g., using nanopores.

A differentially methylated region (DMR) is a genomic region (e.g., set of sites) with different DNA methylation status across two or more biological samples. The different DNA methylation status may be defined by the certain difference in methylation index or density, such as but not limited to 1%, 5%, 10%, 20%, 30%, 40%, 50%, 60%, 70%, 80%, 90%, 95%, 99%, etc. A differentially methylated site may be defined in a similar manner.

The term “hypomethylation” can refer to a site or set of sites (e.g., a region) that has below a specified threshold for a methylation level, e.g., at or below 50%, 45%, 40%, 35%, 30%, 25%, or 20% for the methylation level. A site in a genome may be considered unmethylated if the methylation level is below a threshold. The term “hypermethylation” can refer to a site or set of sites (e.g., a region) that has above a specified value for a methylation level, e.g., at or above 95%, 90%, 80%, 75%, 70%, 65%, or 60% for the methylation level. A site in a genome may be considered methylated if the methylation level is greater than a threshold.

A “relative frequency” (also referred to just as “frequency”) may refer to a proportion (e.g., a percentage, fraction, or concentration). In particular, a relative frequency of a particular nucleotide motif (e.g., A, CG, TAG, etc.) or multiple such motifs can provide a proportion of cell-free DNA fragments that have that particular motif or combination of motifs.

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A normalized amount, e.g., a relative frequency, is an example of a parameter.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

A “separation value” corresponds to a difference or a ratio involving two values, e.g., two fractional contributions or two methylation levels. A separation value is an example of a parameter. The separation value could be a simple difference or ratio. As examples, a direct ratio of x/y is a separation value, as well as x/(x+y). Other examples are y/x and y/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio, e.g., (x−y)/(x+y). A separation value can be compared to a threshold to determine whether the separation between the two values is statistically significant. A separation value is an example of a relative amount. A separation value can be compared to a threshold to determine whether the separation between the two values is statistically significant.

The term “classification” as used herein refers to any number(s) or other characters(s) that are associated with a particular property of a sample. For example, a “+” symbol (or the word “positive”) could signify that a sample is classified as being derived from a subject having a pathology. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample, or tissue fraction can refer to the fractional concentration of DNA from one or more particular tissue(s), e.g., from a transplant organ.

A “calibration sample” can correspond to a biological sample whose fractional concentration of clinically-relevant DNA (e.g., tissue-specific DNA fraction) is known or determined via a calibration method, e.g., using an allele specific to the tissue, such as in transplantation whereby an allele present in the donor's genome but absent in the recipient's genome can be used as a marker for the transplanted organ. As another example, a calibration sample can correspond to a sample from which end motifs can be determined. A calibration sample can be used for both purposes.

A “calibration data point” includes a “calibration value” and a measured or known fractional concentration of the clinically-relevant DNA (e.g., DNA of particular tissue type). The calibration value can be determined from relative frequencies (e.g., an aggregate value) as determined for a calibration sample, for which the fractional concentration of the clinically-relevant DNA is known. The calibration data points may be defined in a variety of ways, e.g., as discrete points or as a calibration function (also called a calibration curve or calibration surface). The calibration function could be derived from additional mathematical transformation of the calibration data points. The fractional concentration can be determined in various ways, e.g., using a tissue-specific allele, a tissue-specific methylation value or pattern, and a size distribution of a sample with a known fractional concentration.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer.

A “level of a pathology” can refer to an amount, degree, or severity of a pathology associated with an organism. A heathy state of a subject can be considered a classification of no pathology. The level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of no pathology.

A “machine learning model” (ML model) can refer to a software module configured to be run on one or more processors to provide a classification or numerical value of a property of one or more samples. An ML model can be generated using sample data (e.g., training data) to make predictions on test data. One example is an unsupervised learning model. Another example type of model is supervised learning that can be used with embodiments of the present disclosure. Example supervised learning models may include different approaches and algorithms including analytical learning, statistical models, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The terms “about” and “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or see, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

The present disclosure provides various methods, products, and systems for identifying and analyzing eccDNA remnants, which are linear DNA molecules resulting from in vivo linearization of eccDNA molecules. The eccDNA remnants can be, for example, products of one or more molecular degradation processes that occur within an organism, and are not the results of in vitro processing steps applied to a biological sample from the organism. By examining sequence information for the linear DNA molecules present in a biological sample, eccDNA remnants can be distinguished from other linear DNA molecules of the sample. Characteristics of the molecules classified as eccDNA remnants can be determined, and these characteristics can advantageously provide information related to the biological sample or to the subject from which the sample was derived. For example, the eccDNA remnants of a biological sample can serve as a biomarker for pathologies such as cancer, enabling beneficial new approaches for determining classifications of such pathologies.

Previous technologies for analyzing the linear DNA found in biological samples failed to appreciate that this linear DNA could include eccDNA remnants. As a result, sequencing information obtained from untreated biological samples was presumed to be associated with only DNA that was always linear, and not previously circular. This sequencing data was processed and analyzed accordingly, with those sequence mapping results inconsistent with linear DNA treated as artifacts or errors, and disregarded. The methods, products, and systems provided herein, however, instead recognize the informational importance of these mapping results that were previously considered to have no value. The disclosed techniques apply this previously unused sequence mapping data to classify whether a sequenced DNA molecule is an eccDNA remnant. By identifying and analyzing the eccDNA remnants that earlier approaches did not, the provided methods, products, and systems advantageously provide otherwise absent insights useful for determining properties of the biological sample and its subject source.

Another advantage of the provided methods, products, and systems is that they eliminate operations previously believed to be necessary for obtaining eccDNA information from a biological sample. In earlier approaches not recognizing that linearized eccDNA remnants may be found in a biological sample, processes were applied to break the circular form of eccDNA, for example through cleavage with enzymes, or shearing with sonication. This in vitro eccDNA breakage linearized the circular eccDNA so that it could be sequenced using techniques designed for sequencing linear nucleic acid molecules. In contrast, the disclosed methods, products, and systems do not rely on any procedures or materials for opening circular eccDNA. Rather, the linear DNA molecules already present in a biological sample are analyzed and, because this linear DNA can include eccDNA remnants, information about eccDNA molecules can be determined more directly.

Furthermore, earlier eccDNA analysis techniques also often applied procedures for reducing or removing linear DNA in a sample prior to linearizing the eccDNA of the sample. This reduction or removal of linear DNA could involve, for example, digestion of linear DNA with an exonuclease such as Exonuclease V (ExoV). Such digestion could decrease a background amount of linear DNA in a sample, thereby enriching the sample for its circular eccDNA content. With the currently provided methods, products, and systems, however, this enrichment is not only unnecessary, but also counterproductive, since any digestion of linear DNA in a sample would also affect (e.g., degrade or otherwise remove) the eccDNA remnants, which have a linear form. Because the disclosed methods, products, and systems thus do not utilize materials or operations for reducing or removing linear DNA, or for opening eccDNA, the disclosure provides advantages of simpler approaches benefiting from improved time and cost efficiencies.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

I. eccDNA Remnants

The eccDNA remnants that are a subject of the present disclosure are linear DNA molecules produced from the circular eccDNA molecules of a subject by natural mechanisms of the subject. For example, the eccDNA remnants can be produced by in vivo nucleic acid degradation processes, such as cleavage by nucleases, e.g., plasma nucleases. Endogenous endonucleases such as deoxyribonuclease 1 (DNASE1), deoxyribonuclease 1 like 3 (DNASE1L3), and DNA fragmentation factor subunit beta (DFFB) may cleave eccDNA. This cleavage can occur intracellularly and/or extracellularly, and forms linearized DNA molecules derived from eccDNA. The present disclosure refers to linearized DNA molecules thus derived from previously intact eccDNA as eccDNA remnants. In contrast, eccDNA remnants do not include linearized DNA molecules created from previously intact eccDNA molecules via synthetic or artificial means.

Different endonucleases prefer to generate DNA molecules with different sizes and end motifs (Serpas L et al., Proc. Natl. Acad. Sci. USA 116, (2019):641; Jiang P et al., Cancer Discov. 10, (2020): 664; Han DSC et al., Am. J. Hum. Genet. 106, (2020): 202), and their enzymatic activities can vary across different tissues. Hence, the analysis of molecular properties of eccDNA remnants generated in vivo can provide information related to their tissues of origin. Additional informative molecular properties of eccDNA remnant molecules include, but are not limited to, amounts, sizes, motifs, jagged ends, and others. These eccDNA properties provide a new data source for determining properties of a subject, such as a classification of a pathology of the subject. As one example, analysis of eccDNA remnant circulating in the plasma of a patient can be used for non-invasive cancer detection. Furthermore, the analysis of eccDNA remnants can allow for determining a fractional concentration of clinically relevant DNA in a biological sample. This clinically relevant DNA could include or consist of, for example, DNA from a tumor, a transplant, or a fetus.

II. Identifying eccDNA Remnants

In some aspects, the present disclosure provides various methods for identifying eccDNA remnants. The methods can be used to, for example, classify whether a linear DNA molecule (e.g., a cell-free DNA linear DNA molecules) is the product of an opening (e.g., an in vivo cleavage) of a circular DNA molecule (e.g., an eccDNA molecule). The methods can thus distinguish eccDNA remnants in a sample from other linear DNA molecules in the sample. This differentiation between eccDNA remnants and other linear DNA molecules relies on the structural and compositional similarities and differences between eccDNA remnants, eccDNA molecules, and linear DNA molecules that are not eccDNA remnants.

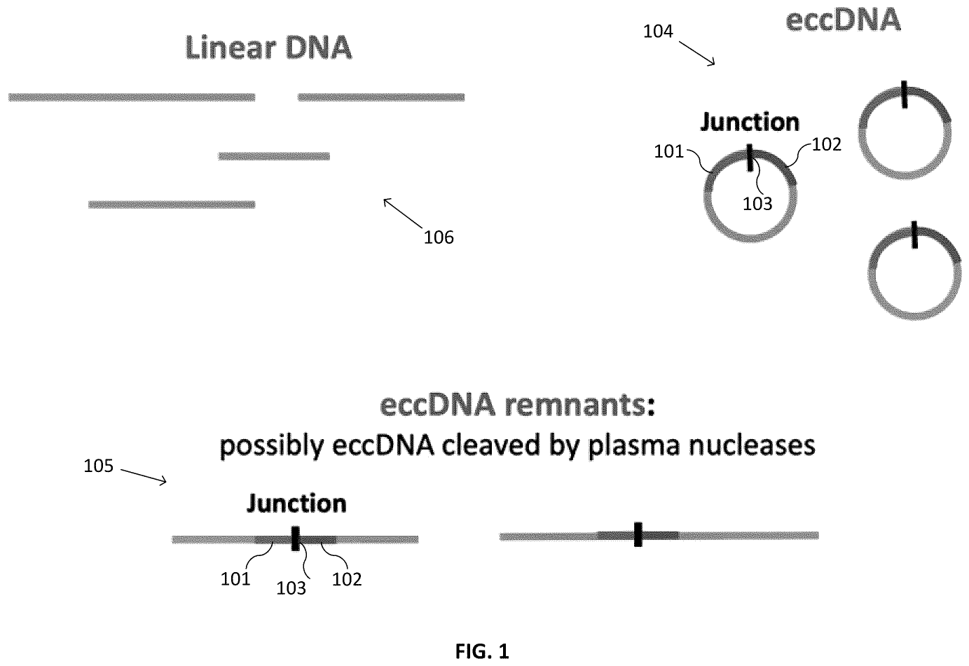

As shown in the illustration of FIG. 1, eccDNA remnants are molecules sharing a linear form with other linear DNA molecules, but also including a junction site characteristic of eccDNA. Because eccDNA is a circularized form of previously linear DNA, e.g., a linear region of genomic DNA, eccDNA molecules contain a junction at the site where the previously separated ends of this linear DNA become adjacent to one another. For example, in FIG. 1, previously separated end sequences 101 and 102 are adjacent to one another at junction 103 of eccDNA molecule 104. In the eccDNA remnants 105 formed by cleavage of the eccDNA molecules with plasma nucleases, the previously separated end sequences 101 and 102 remain connected at junction 103. Other linear DNA 106 does not include a similar junction.

### A. Sequencing and Mapping Linear DNA

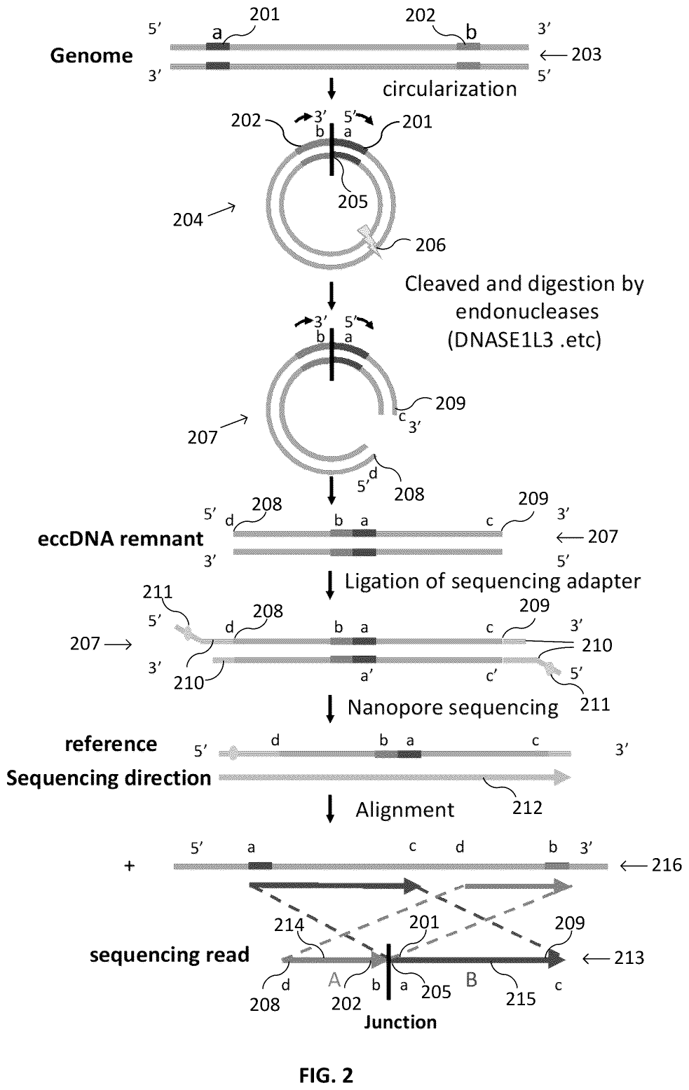

FIG. 2 provides a schematic illustration of concepts underlying the provided method for identifying eccDNA remnants by mapping sequence reads associated with linear DNA molecules. In the exemplary illustration of FIG. 2, sequence “a” 201 and sequence “b” 202 of genome 203 are separated from one another in a genomic region, i.e., a chromosomal region. The genomic region spanning and including sequences “a” and “b” can form eccDNA 204 through a circularization reaction that naturally occurs within a subject. Following this circularization, sequences “a” and “b” are immediately adjacent to one another in the eccDNA molecule, and together form circular junction locus 205.

The eccDNA molecule may subsequently be cleaved in vivo via digestion by an enzyme, e.g., an endonuclease, that is active within the subject. The endonuclease can be, for example, deoxyribonuclease 1 (DNASE1), deoxyribonuclease-1-like 1 (DNASE1L1), deoxyribonuclease-1-like 2 (DNASE1L2), deoxyribonuclease-1-like 3 (DNASE1L3), deoxyribonuclease 2 (DNASE2), endonuclease G (ENDOG), DNA fragmentation factor subunit beta (DFFB), or another endonuclease having activity against circular DNA. The endonuclease can cleave the eccDNA at a cleavage site 206. This opening of the circular DNA molecule at the cleavage site produces a linear DNA molecule 207 having 5′ end sequence “d” 208 and 3′ end sequence “c” 209, where this linear DNA molecule is referred to herein as an eccDNA remnant. In some instances, the cleavage of the eccDNA generates an eccDNA remnant having a shorter size than that of the original eccDNA molecule. In other instances, the cleavage generates an eccDNA remnant having the same size as that of the original eccDNA molecule. Because the eccDNA remnants produced by a subject can circulate in the plasma of the subject, a biological sample of the subject that includes the subject's plasma can contain these eccDNA remnants.

The eccDNA remnants in a biological sample from a subject can be sequenced, for example as also illustrated in FIG. 2, to generate sequence information in the form of one or more sequence reads. In the exemplary workflow shown in FIG. 2, sequencing adapters 210 are ligated to the 5′ end sequence “d” 208 and 3′ end sequence “c” 209 of eccDNA remnant 207. When sequencing is to be performed by nanopore sequencing, the sequencing adapters can include motor proteins 211 for controlling translocation of the eccDNA remnant through a nanopore. Following the ligation of the sequencing adapters to the eccDNA remnant, the resulting construct can be sequenced via, for example, nanopore sequencing. The sequencing direction 212 is from the 5′ end sequence “d” to the 3′ end sequence “c.”

While the illustration of FIG. 2 shows an exemplary workflow in which the one or more sequencing reads corresponding to the eccDNA remnant are generated using nanopore sequencing, other sequencing techniques or alternate approaches can also be used to produce the sequence reads. For example, single-molecule real-time (SMRT) sequencing, sequencing-by-synthesis, ion semiconductor sequencing, and/or chain-termination sequencing can be applied to generate the sequence reads. The sequencing of the eccDNA remnant and other linear DNA molecules of the biological sample can thus involve single-read sequencing or paired-end sequencing. Additionally or alternatively, approaches using hybridization arrays, capture probes, amplifications (e.g., polymerase chain reaction (PCR), linear amplification using a single primer, or isothermal amplification), and/or biophysical measurements (e.g., mass spectrometry) can be used to determine sequence information associated with the eccDNA remnant.

The one or more sequence reads generated by sequencing the eccDNA remnant 207 generally correspond to at least the 5′ end sequence “d” 208 and the 3′ end sequence “c” 209 of the eccDNA remnant. The sequence read that corresponds to the 5′ end sequence of the eccDNA remnant is referred to herein as a 5′ end sequence read and the sequence read that corresponds to the 3′ end of the eccDNA remnant is referred to herein as a 3′ end sequence read. In some examples, and as is shown in the illustration of FIG. 2, a single sequence read 213 spans the entire eccDNA remnant, and therefore corresponds to both the 5′ end sequence and the 3′ end sequence of the eccDNA remnant. In this case (e.g., nanopore sequencing), the single sequencing read is itself both a 5′ end sequence read and a 3′ end sequence read. In other examples (e.g., paired-end sequencing), no single sequence read spans the entire eccDNA remnant, and the 5′ end sequence read and the 3′ end sequence read are different sequence reads.

In some instances, the one or more sequence reads generated by sequencing the eccDNA remnant are raw sequence reads that are pre-processed according to one or more operations. For example, duplicate reads among the one or more sequence reads can be removed. As another example, sequences related to the sequencing adapters can be removed from the sequence reads. Additionally or alternatively, sequences related to low quality bases on the 3′ end of a sequence read can be similarly removed from the sequence read. Another pre-processing operation can involve selecting and/or reversing a specified number of bases at one or both ends of a sequence read for use in alignment, i.e., mapping. For example, when paired-end sequencing is used to generate sequence reads, reversal of some sequence reads prior to mapping is necessary to transform those reads to have sequence orientations matching those of the sequenced linear DNA molecule.

The provided methods for identifying eccDNA remnants generally involve mapping the 5′ end sequence and the 3′ end sequence of the eccDNA remnant to a reference genome. Typically, the reference genome is a reference genome characteristic of the subject from which the eccDNA remnant has been derived. For example, when the eccDNA remnant is a component of a biological sample from a human subject, the reference genome used for mapping of the end sequence reads can be a human reference genome, e.g., hg19. The mapping of the end sequence reads to the reference genome can involve use of alignment techniques such as Bowtie 2 (Langmead et al., Nat. Methods 9, (2012): 357). Other alignment techniques can also be used for the mapping. In some instances, the alignment procedure used for the sequence end mapping can involve a requirement for that the alignment satisfy a predetermined mapping quality condition or threshold. For example, the mapping of the 5′ end sequence and/or the 3′ end sequence can be required to have a mapping quality that is greater than 30, indicating that the probability of this end sequence mapping to a location other than the reported location is 10\hat{ }(−3)=0.1%. In other examples, the mapping qualities for the 5′ end sequence and/or the 3′ end sequence can be required to be greater than 10, e.g., greater than 15, greater than 20, greater than 25, greater than 35, greater than 40, greater than 45, or greater than 50.

In some cases, and as illustrated in FIG. 2, one or both of the 5′ end sequence read and the 3′ end sequence read can include the junction 205 of the eccDNA remnant. In such instances, the end sequence read containing the junction will include two segments that are separated by the junction, and the mapping of these segments to the reference genome will show a discontinuity at the junction. For example, in the mapping illustrated in FIG. 2, sequencing read 213 includes segment “A” 214 and segment “B” 215. Segment “A” of the sequencing read spans the portion of the eccDNA remnant from 5′ end sequence “d” 208 to sequence “b” 202, which forms the 5′ portion of junction locus 205. Segment “B” of the sequencing read spans the portion of the eccDNA remnant from sequence “a” 201, which forms the 3′ portion of the junction locus, to 3′ end sequence “c” 209. When segments “A” and “B” of the sequencing read are aligned to reference genome 216, the mapping results indicate a discontinuity at the junction locus, such that the mapping of segment “B” is not immediately adjacent to and following the mapping of segment “A.” Characteristics of this discontinuous mapping, and its use in identifying eccDNA remnants, are described in more detail in Section II.B. In some instances, the mapping of different junction-separated segments of a sequence read to a reference genome can be required to have a mapping quality that is greater than 10, e.g., greater than 15, greater than 20, greater than 25, greater than 30, greater than 35, greater than 40, greater than 45, or greater than 50.

In some examples, a junction locus of a sequencing read is identified by a process that includes determining that two adjacent segments of the sequencing read optimally align to the reference genome in a discontinuous fashion. After such a determination, the “splitting site” separating these segments in the sequencing read can be identified. This splitting site then corresponds to the junction site or locus of the original eccDNA molecule that was the source of the eccDNA remnant.

B. Classifying eccDNA Based on Linear DNA Mapping

After sequence reads corresponding to a linear DNA molecule have been received, and the 5′ end sequence and the 3′ end sequence of the linear DNA molecule have been mapped to a reference genome, the mapping results can used according to the provided methods to classify whether the linear DNA molecule is an eccDNA remnant. Generally, multiple criteria are used to determine if the mapping results indicate that a linear DNA molecule is an eccDNA remnant. If a linear DNA molecule is an eccDNA remnant, then the mapped genomic coordinate of the 5′ end sequence will be larger than the mapped genomic coordinate of the 3′ end sequence. Relatedly, in many examples the 5′ end sequence and 3′ end sequence of an eccDNA remnant will map to the same chromosome of the genome, while in some other examples, the 5′ end sequence and 3′ end sequence of an eccDNA remnant will map to different chromosomes of the genome.

Additionally, if a linear DNA molecule is an eccDNA remnant, then the mapped genomic orientation of the 5′ end sequence will be identical to the mapped genomic orientation of the 3′ end sequence. Accordingly, in some embodiments, the provided method classifies a linear DNA molecule as being an eccDNA remnant if both: (1) the genomic coordinate of the 5′ end sequence is larger than the genomic coordinate of the 3′ end sequence, and (2) the genomic orientation of the 5′ end sequence is identical to the genomic orientation of the 3′ end sequence.

1. Mapped Genomic Coordinates Indicative of eccDNA Remnants

The genomic coordinate of a particular sequence or sequence segment refers to the location to which the sequence or segment maps in a reference genome. These genomic coordinate locations can be assigned numerical values that increase in a direction from 5′ coordinate locations to 3′ coordinate locations. Accordingly, if a first sequence maps to a location in a reference genome that is downstream, in a 5′ to 3′ direction, of the location to which a second sequence maps, then the first sequence has a larger genomic coordinate than the second sequence. Conversely, the first sequence in this scenario has a smaller genomic coordinate than the second sequence because the first sequence maps to a location upstream of the location of the second sequence in the reference genome.

FIG. 2 provides an example of sequence mapping results with genomic coordinates indicating that a linear DNA molecule is an eccDNA remnant. In the illustrated example, the 5′ end sequence “d” 208 of sequencing read 213 maps to corresponding sequence “d” in the reference genome 216. Similarly, the 3′ end sequence “c” 209 of the sequencing read maps to corresponding sequence “c” in the reference genome. Because sequence “d” is downstream of sequence “c” in the reference genome in a 5′ to 3′ direction, the mapped genomic coordinate of 5′ end sequence “d” of linear DNA molecule 207 is greater than the mapped genomic coordinate of 3′ end sequence “c” of the linear DNA molecule.

Alternative or additional mapping criteria related to genomic coordinates can be used in cases where at least one received sequencing read associated with a linear DNA molecule includes an identified junction. For example, if a 5′ end sequence read was determined to contain a junction locus, then the read would necessarily include a 5′ end sequence, a sequence forming the 5′ portion of the junction locus, and a sequence forming the 3′ portion of the locus. Referring to the example illustrated in FIG. 2, the sequencing read 213 includes the 5′ end sequence “d” 208, sequence “b” which forms the 5′ portion of junction 205, and sequence “a” which forms the 3′ portion of junction 205. In such cases, the sequence forming the 3′ portion of the junction (e.g., sequence “a”) maps to a genomic coordinate that is less than the genomic coordinate to which the sequence forming the 5′ portion of the junction (e.g., sequence “b”) maps. Additionally, the sequence forming the 3′ portion of the junction (e.g., sequence “a”) maps to a genomic coordinate that is less than the genomic coordinate to which the 5′ end sequence (e.g., sequence “d”) maps.

As another example, if a 3′ end sequence read was determined to contain a junction locus, then the read would necessarily include a 3′ end sequence, a sequence forming the 5′ portion of the junction locus, and a sequence forming the 3′ portion of the locus. Referring again to the example illustrated in FIG. 2, the sequencing read 213 includes the 3′ end sequence “c” 209, sequence “b” which forms the 5′ portion of junction 205, and sequence “a” which forms the 3′ portion of junction 205. In such cases, the sequence forming the 5′ portion of the junction (e.g., sequence “b”) maps to a genomic coordinate that is greater than the genomic coordinate to which the sequence forming the 3′ portion of the junction (e.g., sequence “a”) maps. Additionally, the sequence forming the 5′ portion of the junction (e.g., sequence “b”) maps to a genomic coordinate that is greater than the genomic coordinate to which the 3′ end sequence (e.g., sequence “c”) maps.

Also, where at least one received sequencing read associated with a linear DNA molecule includes an identified junction, the mapped genomic coordinates of segments on either side of this identified junction can further indicate that the linear DNA molecule is an eccDNA remnant. In particular, for such a sequencing read the segment that corresponds to a more 5′ portion of the linear DNA molecule will map to a greater genomic coordinate than the segment of the sequencing read corresponding to a more 3′ portion of the linear DNA molecule. As an illustration, for sequencing read 213 of FIG. 2, segment “A” 214 corresponds to a more 5′ portion of linear DNA molecule 207, while segment “B” 215 corresponds to a more 3′ portion of the linear DNA molecule. Mapping of these segments to reference genome 216 shows that segment “A” has a larger genomic coordinate than segment “B,” providing further evidence that the linear DNA molecule is an eccDNA remnant.

2. Mapped Genomic Orientations Indicative of eccDNA Remnants

The genomic orientation of a particular sequence or sequence segment refers to the direction with which the sequence segment maps to a reference genome. For example, when a sequence corresponding to a 5′ to 3′ region of a sequenced DNA molecule optimally aligns to a region of a reference genome in the same 5′ to 3′ direction, the sequence can be referred to as having a forward or positive genomic orientation. Conversely, when a sequence corresponding to a 5′ to 3′ region of a sequenced DNA molecule optimally aligns to a region of a reference genome in an opposite 3′ to 5′ direction, the sequence can be referred to as having a reverse or negative genomic orientation.

FIG. 2 provides an example of sequence mapping results with genomic orientations indicating that a linear DNA molecule is an eccDNA remnant. In the illustrated example, the 5′ end sequence “d” 208 of sequencing read 213 maps to reference genome 216 with a forward genomic orientation. Similarly, the 3′ end sequence “c” 209 of the sequencing read also maps to the reference genome with a forward orientation. These identical genomic orientations, together with the genomic coordinate information described in Section II.B.1, can be used to identify the linear DNA molecule 207 as being an eccDNA remnant.

Alternative or additional mapping criteria related to genomic orientations can be used in cases where at least one received sequencing read associated with a linear DNA molecule includes an identified junction. For example, if a 5′ end sequence read was determined to contain a junction locus, then the read would necessarily include a 5′ end sequence, a sequence forming the 5′ portion of the junction locus, and a sequence forming the 3′ portion of the locus. Referring to the example illustrated in FIG. 2, the sequencing read 213 includes the 5′ end sequence “d” 208, sequence “b” which forms the 5′ portion of junction 205, and sequence “a” which forms the 3′ portion of junction 205. In such cases, the sequence forming the 3′ portion of the junction (e.g., sequence “a”) has a genomic orientation identical to that of the sequence forming the 5′ portion of the junction (e.g., sequence “b”). Additionally, the sequence forming the 3′ portion of the junction (e.g., sequence “a”) has a genomic orientation that is identical to that of the 5′ end sequence (e.g., sequence “d”).

As another example, if a 3′ end sequence read was determined to contain a junction locus, then the read would necessarily include a 3′ end sequence, a sequence forming the 5′ portion of the junction locus, and a sequence forming the 3′ portion of the locus. Referring again to the example illustrated in FIG. 2, the sequencing read 213 includes the 3′ end sequence “c” 209, sequence “b” which forms the 5′ portion of junction 205, and sequence “a” which forms the 3′ portion of junction 205. In such cases, the sequence forming the 5′ portion of the junction (e.g., sequence “b”) has a genomic orientation identical to that of the sequence forming the 3′ portion of the junction (e.g., sequence “a”). Additionally, the sequence forming the 5′ portion of the junction (e.g., sequence “b”) has a genomic orientation that is identical to that of the 3′ end sequence (e.g., sequence “c”).

Also, where at least one received sequencing read associated with a linear DNA molecule includes an identified junction, the mapped genomic orientations of segments on either side of this identified junction can further indicate that the linear DNA molecule is an eccDNA remnant. In particular, for such a sequencing read the segment that corresponds to a more 5′ portion of the linear DNA molecule will have a genomic orientation identical to that of a more 3′ portion of the linear DNA molecule. As an illustration, for sequencing read 213 of FIG. 2, segment “A” 214 corresponds to a more 5′ portion of linear DNA molecule 207, while segment “B” 215 corresponds to a more 3′ portion of the linear DNA molecule. Mapping of these segments to reference genome 216 shows that segment “A” has the same forward genomic orientation of segment “B,” providing further evidence that the linear DNA molecule is an eccDNA remnant.

3. Mapping Results not Indicative of eccDNA Remnants

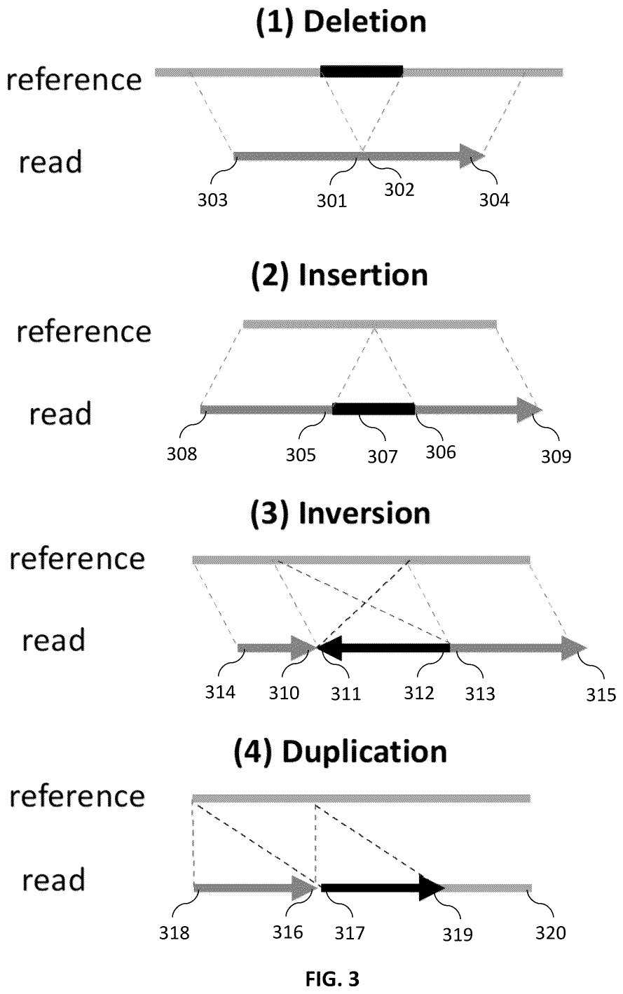

The particular sequence mapping results described in Sections II.B.1 and II.B.2 can be used according to the provided methods to classify a linear DNA molecule as an eccDNA remnant, because different sequence mapping results are seen with sequence reads associated with linear DNA molecules that are not eccDNA remnants. For example, the mapping features of eccDNA remnants are distinct from mapping configurations other linear DNA structural variants that also exhibit mapping discontinuities. These alternate linear structural variants include, for example, the deletions, insertions, inversions, and duplications of genomic DNA segments illustrated in FIG. 3 (van Belzen IAM, Schönhuth A, Kemmeren P & Hehir-Kwa, npj Precis. Oncol. 5, (2021): 1).

In the case of a linear DNA molecule resulting from the deletion of a genomic DNA section, the mapping of a sequence read of the linear DNA molecule will show a discontinuity if the read includes sequences adjacent to the deleted genomic section. In this case, adjacent sequence segments in the read (e.g., sequence segments 301 and 302 in FIG. 3) will optimally align to genomic coordinates that are not adjacent to one another, as with mapping associated with eccDNA remnants. However, unlike with eccDNA mapping results, the genomic coordinate of the 5′ end sequence (e.g., end sequence 303) will be smaller, not larger, than the genomic coordinate of the 3′ end sequence (e.g., end sequence 304). This difference can be used to differentiate linear eccDNA remnants from linear DNA molecules resulting from genomic deletions.

In the case of a linear DNA molecule resulting from the insertion of a sequence, the mapping of a sequence read of the linear DNA molecule will show a discontinuity if the read includes sequences adjacent to and within the inserted sequence. In this case, one or more sequence segments (e.g., sequence segment 305 and/or 306 in FIG. 3) will optimally align to the reference genome, whereas an adjacent sequence segment (e.g., sequence segment 307) will not align. Relatedly, the sequence read may also include non-adjacent sequence segments (e.g., sequence segments 305 and 306) that map to adjacent sequences in the reference genome. In the insertion case, however, the genomic coordinate of the 5′ end sequence (e.g., end sequence 308) will be smaller, not larger, than the genomic coordinate of the 3′ end sequence (e.g., end sequence 309). This difference can be used to differentiate linear eccDNA remnants from linear DNA molecules resulting from insertions.

In the case of a linear DNA molecule resulting from inversion of a genomic DNA section, the mapping of a sequence read of the linear DNA molecule will show a discontinuity if the read includes sequences adjacent to and within the inverted genomic section. In this case, adjacent sequence segments in the read (e.g., sequence segments 310 and 311, or 312 and 313, in FIG. 3) will optimally align to genomic coordinates that are not adjacent to one another, as with mapping associated with eccDNA remnants. However, these sequence segment pairs (e.g., sequence segments 310 and 311, or 312 and 313) will have genomic orientations that are opposite to one another as a result of the inversion. In contrast, sequence segments from reads of eccDNA remnants do not exhibit dissimilar genomic orientations. Further, for the read of the linear DNA molecule having an inversion, the genomic coordinate of the 5′ end sequence (e.g., end sequence 314) will be smaller, not larger, than the genomic coordinate of the 3′ end sequence (e.g., end sequence 315). This property will hold even if the linear DNA molecule includes the inversion at the 5′ end of the linear DNA molecule (in which case, for example, the genomic coordinate of sequence segment 311 is less than that of sequence segment 315), or includes the inversion at the 3′ end of the linear DNA molecule (in which case, for example, the genomic coordinate of sequence segment 314 is less than that of sequence segment 312). These differences can be used to differentiate linear eccDNA remnants from linear DNA molecules resulting from genomic inversions.

In the case of a linear DNA molecule resulting from duplication of a genomic DNA section, the mapping of a sequence read of the linear DNA molecule will show a discontinuity if the read includes sequences within each duplication. In this case, for example, adjacent sequence segments in the read (e.g., sequence segments 316 and 317 in FIG. 3) will optimally align to genomic coordinates that are not adjacent to one another, as with mapping associated with eccDNA remnants. Unlike for eccDNA remnant sequence reads though, sequence reads from a linear DNA molecule resulting from a duplication can include pairs of sequence segments (e.g., sequence segments 317 and 318, or 316 and 319) that each map to the same genomic coordinate. Further, for the read of the linear DNA molecule having a duplication, the genomic coordinate of the 5′ end sequence (e.g., end sequence 318) will be smaller, not larger, than the genomic coordinate of the 3′ end sequence (e.g., end sequence 320). These differences can be used to differentiate linear eccDNA remnants from linear DNA molecules resulting from genomic duplications.

### C. Method

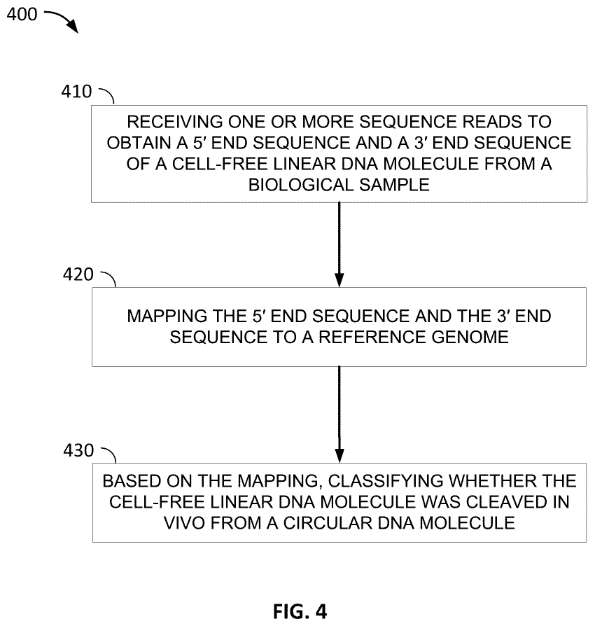

FIG. 4 presents a flowchart of a method 400 for determining if a linear DNA molecule in a biological sample is an eccDNA remnant according to embodiments of the present disclosure. Various examples of method 400 are described in Sections II.A and IIB. Method 400 can be performed partially or entirely using a computer system.

At block 410, one or more sequence reads are obtained, where the one or more sequence reads include a 5′ end sequence of a linear DNA molecule in a biological sample, and a 3′ end sequence of the linear DNA molecule. In some examples, obtaining the sequence reads includes receiving the biological sample. The biological sample can include cell-free DNA. For example, the biological sample can be one that is purified, e.g., to separate out a predominantly cell-free portion, such as plasma. Other pre-processing steps may be performed with the biological sample as well. In some examples, obtaining the sequencing reads includes sequencing the linear DNA molecules present in the sample. The sequencing can be a random sequencing of all the linear DNA molecules in the biological sample, rather than a targeted sequencing of particular molecules, and can be performed using any of the sequencing techniques described in Section II.A. In some instances, the biological sample is a cell-free biological sample and/or the linear DNA molecules are cell-free linear DNA molecules. For example, the biological sample can include or consist of plasma. The linear DNA molecules of the biological sample are naturally found in the biological sample in a linear form, and the provided method does not include operations, such as enzymatic cleavage or mechanical shearing, intended to linearize circular DNA molecules of the biological sample. In some instances, one obtained sequence read includes both the 5′ end sequence and the 3′ end sequence of a linear DNA molecule. In other instances, one obtained sequence read includes the 5′ end sequence of a linear DNA molecule, and another obtained sequence read includes the 3′ end sequence of the linear DNA molecule. The obtained sequence reads can each independently have a length that is at least 25 bp, at least 45 bp, at least 75 bp, at least 150 bp, at least 250 bp, at least 500 bp, at least 1 kb, at least 3 kb, at least 10 kb, at least 30 kb, or at least 100 kb

At block 420, the 5′ end sequence of the linear DNA molecule and the 3′ end sequence of the linear DNA molecule obtained in block 410 are mapped to a reference genome. The mapping can be performed as described in Section II.A, and can involve independently determining an optimal alignment for each of the 5′ end sequence and the 3′ end sequence to the reference genome. In some instances, the alignments are each independently required to satisfy a mapping quality condition, for example, having a mapping quality that is at least 20, at least 25, at least 30, at least 35, at least 40, at least 45, or at least 50. In some cases, the mapping of the end sequences includes determining a genomic coordinate for each of the end sequences. Additionally or alternatively, the mapping of the end sequences can include determining a genomic orientation for each of the end sequences. In some examples, the provided method also includes additionally mapping one or more sequences of the linear DNA molecule other than the 5′ end sequence and the 3′ end sequence, and determining the genomic coordinates and/or genomic orientations of these additionally mapped sequences. In some instances, the mapping further includes identifying if a sequence read includes a junction locus, and optionally determining the genomic coordinate of this junction.

At block 430, the linear DNA molecule is classified, based on the mapping in block 420, according to whether or not the linear DNA molecule was cleaved in vivo from a circular DNA molecule, i.e., whether or not the linear DNA molecule is an eccDNA remnant. The classifying of the linear DNA molecule can be performed as described in Section II.B. For instance, the classifying can include comparing the genomic coordinate of the 5′ end sequence of the linear DNA molecule to the genomic coordinate of the 3′ end sequence of the linear DNA molecule. Additionally or alternatively, the classifying can include comparing the genomic orientation of the 5′ end sequence to the genomic orientation of the 3′ end sequence. As one example, the linear DNA molecule can be classified as an eccDNA remnant if (1) the genomic coordinate of the 5′ end sequence is larger than the genomic coordinate of the 3′ end sequence, and (2) the genomic orientation of the 5′ end sequence is identical to the genomic orientation of the 3′ end sequence. In some cases, the classifying can include comparisons of genomic coordinates and/or genomic orientations of mapped sequences other than or in addition to the 5′ end sequence and the 3′ end sequence.

III. Analyzing eccDNA Remnants to Determine Property

In some aspects, the present disclosure also provides various methods for determining a property of a biological sample, or of a subject from whom the biological sample was obtained, where the property is determined by analyzing eccDNA remnants in the biological sample. Because previous approaches for determining a property of a biological sample or subject did not consider or analyze eccDNA remnants present in a biological sample, the methods disclosed herein advantageously provide a new source of information that can be useful in, for example, identifying a health status of a subject, and/or recognizing, classifying, or treating pathologies of the subject.

The provided methods for determining a property of a biological sample or subject generally include classifying all, or at least a portion of, the linear DNA molecules of the biological sample according to whether each of these linear DNA molecules is an eccDNA remnant. In this way a set of linear DNA molecules classified as eccDNA remnants can be identified. The classifying of each linear DNA molecule can be performed as described in Section II. The number of linear DNA molecules classified can be, for example, at least 100, at least 300, at least 1000, at least 3000, at least 10,000, at least 30,000, at least 100,000, at least 300,000, at least 1,000,000, or at least 3,000,000. Once the set of classified eccDNA remnants has been identified, the eccDNA remnants of the set can be analyzed, for example to determine a collective value of the members of the set. In some instances, the collective value can be compared to a reference value to determine the property of the biological sample or subject. In some instances, the determining of the property of the biological sample or subject involves comparing the collective value can to a threshold and determining if the collective value exceeds or falls below the threshold.

### A. Count

In some examples, the provided methods for determining a property of a biological sample, or from a subject from whom the biological sample was obtained, include determining a count of the linear DNA molecules of the biological sample that are classified as being eccDNA remnants. The count can be a raw value indicating the absolute number, i.e., numerical amount, of members in a set of all classified eccDNA remnants from the biological sample. Alternatively, the count can be a processed value. For example, the count can be a normalized value indicating a relative number of members in a set of all classified eccDNA remnants from the biological sample. In some instances, the count is a relative number arrived at by dividing the absolute number of classified eccDNA remnants in the biological sample, by the absolute number of the plurality of linear DNA molecules for which sequence reads were received and mapped. In this way the eccDNA remnant count can be normalized with respect to a count of the linear DNA molecules in the plurality of linear DNA molecules. In some examples, such normalized counts can be reported in terms of eccDNA Remnants Per Million mapped reads (EPM). Other methods for processing a raw or normalized eccDNA remnant count can additionally or alternatively include dividing the count by the volume of the biological sample, thereby transforming the raw or normalized count into a concentration value.

The provided methods can further include using the determined count, e.g., the determined normalized count, to determine a property of the biological sample or of the subject. For example, the determined count of eccDNA remnants for the particular biological sample can be compared to a previously determined count for a reference sample. Likewise, the determined count of eccDNA remnants can be compared to a reference value. The determined count can also be compared to two or more reference values. In some examples, two or more reference values are used to create a standard curve that is fit to the reference values, and the determined count is compared to the standard curve. In other examples, the determined count of eccDNA remnants is compared to a threshold value, where a particular property of the biological sample or of the subject is indicated if the determined count is less than or greater than the threshold value.

### B. Size Distribution

In some examples, the provided methods for determining a property of a biological sample, or from a subject from whom the biological sample was obtained, include determining a size distribution of the linear DNA molecules of the biological sample that are classified as being eccDNA remnants. This size distribution also or alternatively indicates a deduced size distribution of the original eccDNA molecules corresponding to the eccDNA remnants. The size distribution can include information about the sizes (e.g., estimated sizes) of all, or at least a portion of, the linear DNA molecules classified as being eccDNA molecules. In some instances, the size distribution includes one or more counts (e.g., absolute counts or relative or normalized counts) of the eccDNA remnants having a size (e.g., estimated size) that is greater than and/or less than one or more threshold values. For example, the size distribution can include absolute and/or relative counts of eccDNA remnants or original eccDNA molecules having estimated sizes that are greater than 100 bp, greater than 300 bp, greater than 1 kb, greater than 3 kb, greater than 10 kb, greater than 30 kb, and/or greater than 100 kb. The size distribution can additionally or alternatively include absolute and/or relative counts of eccDNA remnants or original eccDNA molecules having estimated sizes that are less than 100 kb, less than 30 kb, less than 10 kb, less than 1 kb, less than 300 bp, or less than 100 bp. In some instances, the size distribution includes one or more percentages of the eccDNA remnants or original eccDNA molecules, where each of the one more percentages has a size within a predetermined range of sizes, and where each of the ranges has a lower bound, an upper bound, or both. For example, the size distribution can include a size-frequency distribution.

In some examples at least one sequence read for each of all, or at least a portion of, the linear DNA molecules classified as being eccDNA remnants includes both the 5′ end sequence and the 3′ end sequence of the linear DNA molecules. For instance, in the example illustrated in FIG. 2, sequence read 213 includes both the 5′ end sequence 208 and the 3′ end sequence 209 of the linear DNA molecule 207. In such cases, the sequence read can be presumed to include the entire sequence of the linear DNA molecule (e.g., the eccDNA remnant), and the length of the sequence read will be equal to the length of the linear DNA molecule. Therefore, if a sequence read includes the 5′ end sequence and the 3′ end sequence of a linear DNA molecule classified as an eccDNA remnant, then the size of the eccDNA remnant can be determined by measuring the length of the sequencing read, i.e., calculating the distance between the 5′ end of the 5′ end sequence and the 3′ end of the 3′ end sequence.

In some examples, at least one sequence read for each of all, or at least a portion of, the linear DNA molecules classified as being eccDNA remnants includes a junction, i.e., a site within the sequence at which nucleotides at two separated genomic locations are immediately adjacent to one another. For instance, in the example illustrated in FIG. 2, sequence read 213 includes the junction 205 of the eccDNA molecule 204, where the nucleotides of sequence segments 202 and 201 are immediately adjacent to one another in the sequence read, but map to separated genomic coordinates in the reference genome 216. In such cases, the nucleotides on the two sides of the junction represent the 5′ end and the 3′ end of the genomic sequence that was circularized to form the eccDNA molecule. As a result, the distance between these nucleotides in the reference genome is equal to the length of the original eccDNA molecule. Therefore, if a sequence read of a linear DNA molecule classified as an eccDNA remnant includes a junction, then the size of the original eccDNA molecule can be determined by calculating the distance between the genomic locations, i.e., genomic coordinates, of the nucleotides forming the 5′ and 3′ portions of the junction.

In some examples, no sequence read for a particular linear DNA molecule classified as being an eccDNA remnant includes either a junction, or both the 5′ end sequence and the 3′ end sequence of the linear DNA molecule. In such cases, the length of the linear DNA molecule can be presumed to be greater than the combined lengths of the read including the 5′ end sequence (i.e., the 5′ end sequence read) and the read including the 3′ end sequence (i.e., the 3′ end sequence read) minus any overlapping region shared by the 5′ end sequence and the 3′ end sequence. Therefore, a size of an eccDNA remnant or original eccDNA molecule can be estimated by determining the length of the 5′ end sequence read of the eccDNA remnant, determining the length of the 3′ end sequence read of the eccDNA remnant, summing these two lengths, and approximating that the size of the eccDNA remnant is greater than this sum minus the length of any overlapping region shared by the 5′ end sequence and the 3′ end sequence. In some examples of the provided methods, such estimates and approximations are not used in the determining of the size distribution, and the size distribution is instead determined only using data from sequence reads that include a junction, and optionally further include both the 5′ end sequence and the 3′ end sequence of the same linear DNA molecule.

The provided methods can further include using the determined size distribution to determine a property of the biological sample or of the subject. For example, the determined size distribution of eccDNA remnants or deduced size distribution of original eccDNA molecule for the particular biological sample can be compared to a previously determined size distribution for a reference sample. Likewise, the determined size distribution of eccDNA remnants or deduced size distribution of original eccDNA molecules can be compared to a reference value. The determined size distribution can also be compared to two or more reference values. In some examples, two or more reference values are used to create a standard curve that is fit to the reference values, and the determined size distribution is compared to the standard curve. In other examples, the determined size distribution of eccDNA remnants or deduced size distribution of eccDNA molecules is compared to a threshold value, where a particular property of the biological sample or of the subject is indicated if the determined size distribution is less than or greater than the threshold value.

### C. Genomic Distribution

In some examples, the provided methods for determining a property of a biological sample, or from a subject from whom the biological sample was obtained, include determining the genomic distribution of the linear DNA molecules of the biological sample that are classified as being eccDNA remnants. In these examples, the methods generally include identifying a set of eccDNA remnants according to the classification techniques described in Section II.B. The methods further include determining whether each eccDNA remnant of the set of eccDNA remnants belongs to a subset that maps to one or more regions within a class of genomic elements of the reference genome. In some cases, an eccDNA remnant is classified as belonging to the subset when the start position of the eccDNA remnant maps to the particular class of genomic elements. The class can include any one or more types of genomic elements generally known to occur in genomic DNA. For example, the class can include 5′ untranslated regions, 3′ untranslated regions, exons, introns, regions 2 kb upstream of genes (Gene2kbU), regions 2 kb downstream of genes (Gene2kbD), CpG islands, regions 2 kb upstream of CpG islands (CGI2kbU), regions 2 kb downstream of CpG islands (CGI2kbD), Alu repeat regions, or any combination of these types of genomic elements.

The methods of these examples further include determining a count of the subset of the eccDNA remnants. The count can be an absolute count, a relative count, or a normalized count. In some instances, the count is normalized to generate a normalized genomic distribution of the eccDNA remnants. The normalization can relate the count to the theoretical distribution of all genomic DNA that belongs to the particular class of genomic elements. Accordingly, the normalization process can include determining the percentage of the genome that is covered by the class, and dividing the percentage of eccDNA remnants mapping to the class by the percentage of the genome covered by the class.

The provided methods can further include using the normalized genomic distribution to determine a property of the biological sample or of the subject. For example, the determined normalized genomic distribution of eccDNA remnants for the particular biological sample can be compared to a previously determined normalized genomic distribution for eccDNA remnants from a reference sample. Likewise, the determined normalized genomic distribution of eccDNA remnants can be compared to a reference distribution. The determined normalized genomic distribution can also be compared to two or more reference distributions. In some examples, two or more reference distributions are used to create a standard curve that is fit to the reference distributions, and the determined normalized genomic distribution is compared to the standard curve. In other examples, the determined normalized genomic distribution of eccDNA remnants is compared to a threshold distribution, where a particular property of the biological sample or of the subject is indicated if the determined normalized genomic distribution is less than or greater than the threshold distribution.

### D. Nucleotide Motif Frequency

In some examples, the provided methods for determining a property of a biological sample, or from a subject from whom the biological sample was obtained, include determining the frequency of one or more nucleotide motif patterns occurring for the linear DNA molecules of the biological sample that are classified as being eccDNA remnants. In these examples, the methods generally include identifying a set of eccDNA remnants according to the classification techniques described in Section II.B. As part of these classification techniques, the start positions for each eccDNA remnant (i.e., the genomic position of the upstream edge of the eccDNA remnant) and end positions for each remnant (i.e., the genomic position of the downstream edge of the eccDNA remnant) can be determined. Since these start and end positions indicate where the excision of these fragments from the genome occurred prior to eccDNA formation, DNA sequences (i.e., eccDNA sequences and/or genome sequences) within a specified distance of the start and end positions can include recurrent nucleotide motif signatures associated with the fragment excision.

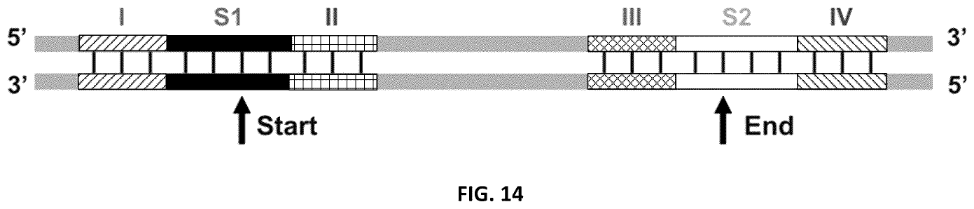

FIG. 14 illustrates one example of the Start position and the End position of an eccDNA molecule. In this particular example, both the Start and End positions are flanked by a pair of trinucleotide motifs with 4-bp spacer regions in between. Specifically, trinucleotide motif I is upstream of the eccDNA Start position, trinucleotide motif II is downstream of the eccDNA Start position, and motifs I and II are adjacent to spacer region S1, which includes the Start position. Similarly, trinucleotide motif III is upstream of the eccDNA End position, trinucleotide motif IV is downstream of the eccDNA End position, and motifs III and IV are adjacent to spacer region S2, which includes the End position. Together, the trinucleotide motifs I, II, III, and IV constitute a nucleotide motif pattern.

While the example of FIG. 14 includes a 4-motif pattern of trinucleotides, other configurations of patterns and motifs can be used with the provided methods. For instance, nucleotide motif patterns can each independently include only a single nucleotide motif, or a plurality of nucleotide motifs, e.g., at least two nucleotide motifs, at least three nucleotide motifs, at least four nucleotide motifs, at least six nucleotide motifs, at least seven nucleotide motifs, at least eight nucleotide motifs, at least nine nucleotide motifs, at least ten nucleotide motifs, or more than ten nucleotide motifs. Also, the nucleotide motifs can each independently include various pluralities of nucleotides, such that each motif can independently be a dinucleotide motif, a trinucleotide motif, a tetranucleotide motif, a pentanucleotide motif, a hexanucleotide motif, a septanucleotide motif, an octanucleotide motif, a nonanucleotide motif, a decanucleotide motif, or a larger nucleotide motif.

While in some examples, and as illustrated in FIG. 14, a nucleotide pattern can include multiple nucleotide motifs that together flank both the Start and End positions of an eccDNA fragment, other configurations of nucleotide motifs can be used with the provided method. In some examples, at least one nucleotide motif of a nucleotide motif pattern is upstream of the eccDNA Start position. In some examples, each nucleotide motif of a nucleotide motif pattern is upstream of the eccDNA Start position. In some examples, at least one nucleotide motif of a nucleotide motif pattern is downstream of the eccDNA Start position and upstream of the eccDNA End position. In some examples, each nucleotide motif of a nucleotide motif pattern is downstream of the eccDNA Start position and upstream of the eccDNA End position. In some examples, at least one nucleotide motif of a nucleotide motif pattern is downstream of the eccDNA End position. In some examples, each nucleotide motif of a nucleotide motif pattern is downstream of the eccDNA End position.

In some examples, the method includes determining a frequency of a single nucleotide motif pattern occurring for the set of eccDNA remnants. In other examples, the method includes determining a frequency of a plurality of nucleotide motif patterns occurring for the set of eccDNA patterns. In this latter case, the frequency can be an aggregate or average of multiple frequencies, each frequency corresponding to one of the plurality of nucleotide motif patterns.

Each nucleotide motif is located in a respective segment of either the reference genome (e.g., for segments upstream of the eccDNA Start position or downstream of the eccDNA End position) or of the eccDNA remnant (e.g., for segments downstream of the eccDNA Start position and upstream of the eccDNA End position). Each segment can independently be within a specified distance from the Start position (i.e., a respective 5′ end of an eccDNA remnant of the set of eccDNA remnants) or from the End position (i.e., a respective 3′ end of an eccDNA remnant of the set of eccDNA remnants). The specified distance can be, for example, 1 bp, 2 bp, 3 bp, 4 bp, 5 bp, 6 bp, 7 bp, 8 bp, 9 bp, 10 bp, 15 bp, 20 bp, 25 bp, 30 bp, 40 bp, 50 bp, 60 bp, 80 bp, 100 bp, 150 bp, 200 bp, or larger than 200 bp.

The provided methods can further include using the determined frequency of one or more nucleotide motif patterns occurring for the set of eccDNA remnants to determine a property of the biological sample or of the subject. For example, the determined frequency occurring for eccDNA remnants for the particular biological sample can be compared to a previously determined frequency occurring for eccDNA remnants from a reference sample. Likewise, the determined frequency occurring for eccDNA remnants can be compared to a reference frequency. The determined frequency can also be compared to two or more reference frequencies. In some examples, two or more reference frequencies are used to create a standard curve that is fit to the reference frequencies, and the determined frequency is compared to the standard curve. In other examples, the determined frequency for the set of eccDNA remnants is compared to a threshold frequency, where a particular property of the biological sample or of the subject is indicated if the determined frequency is less than or greater than the threshold frequency.

### E. Methylation Density

In some examples, the provided methods for determining a property of a biological sample, or from a subject from whom the biological sample was obtained, include determining the methylation density of at least a subset of the linear DNA molecules of the biological sample that are classified as being eccDNA remnants. In these examples, the methods generally include identifying a set of eccDNA remnants according to the classification techniques described in Section II.B. The methods generally further include determining sizes for at least a portion of the set of eccDNA remnants, for example by using procedures described in Section IIIB. Based on these determined sizes, a subset of the set of eccDNA remnants is identified, where each eccDNA remnant of the subset independently has a size within a specified range size.

In some examples, each DNA remnant of the subset of eccDNA remnants independently has a size less than a maximum size. The maximum size can be, for example, about 300 bp, about 400 bp, about 500 bp, about 600 bp, about 700 bp, about 800 bp, about 900 bp, about 1000 bp, about 1200 bp, or about 1500 bp. In other examples, each DNA remnant of the subset of eccDNA remnants independently has a size greater than a minimum size. The minimum size can be, for example, about 600 bp, about 700 bp, about 800 bp, about 900 bp, about 1000 bp, about 1200 bp, about 1500 bp, about 2000 bp, about 2500 bp, or about 3000 bp.

The methods of these examples further include determining a methylation status at one or more sited of each eccDNA remnant of the subset of eccDNA remnants. Based on the determined methylation statuses, a methylation density for the subset of eccDNA fragments is then determined. The provided methods can further include using the determined methylation density for the subset to determine a property of the biological sample or of the subject. For example, the determined methylation density for the subset of eccDNA remnants from the particular biological sample can be compared to a previously determined methylation density for eccDNA remnants from a reference sample. Likewise, the determined methylation density for the subset of eccDNA remnants from the sample can be compared to a reference methylation density. The determined methylation density can also be compared to two or more reference methylation densities. In some examples, two or more reference methylation densities are used to create a standard curve that is fit to the reference methylation densities, and the determined methylation density is compared to the standard curve. In other examples, the determined methylation density for the subset of eccDNA remnants from the sample is compared to a threshold methylation density, where a particular property of the biological sample or of the subject is indicated if the determined methylation density is less than or greater than the threshold methylation density.

### F. Method

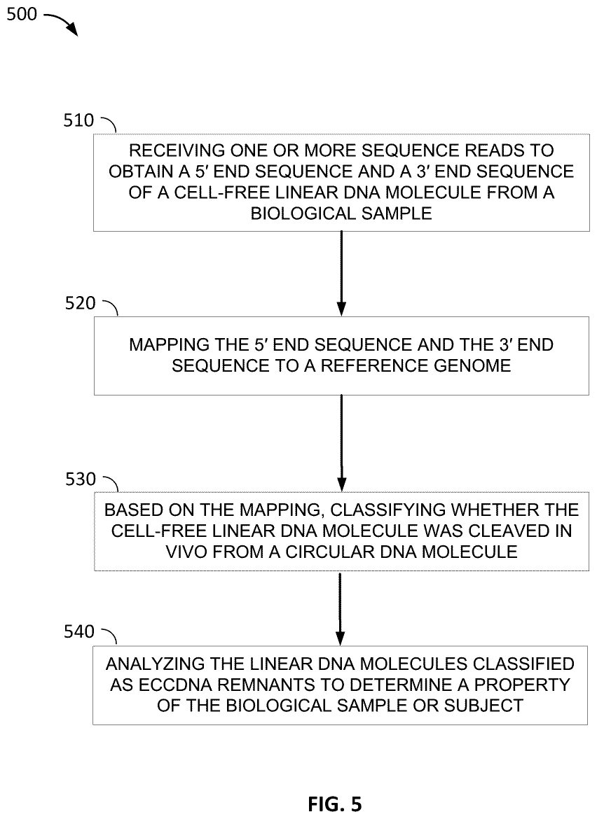

FIG. 5 presents a flowchart of a method 500 for analyzing a biological sample from a subject to determine a property of the biological sample or subject based on an analysis of eccDNA remnants according to embodiments of the present disclosure. Various examples of method 500 are described in Sections III.A and III.B. Method 500 can be performed partially or entirely using a computer system.

At block 510, one or more sequence reads are obtained, where the one or more sequence reads include a 5′ end sequence of a linear DNA molecule in a biological sample, and a 3′ end sequence of the linear DNA molecule. Block 510 can be performed in a similar manner to block 410 of method 400, presented in FIG. 4. As with block 410, in some examples, obtaining the sequence reads includes receiving the biological sample. The biological sample can include cell-free DNA. For example, the biological sample can be one that is purified, e.g., to separate out a predominantly cell-free portion, such as plasma. Other pre-processing steps may be performed with the biological sample as well. In some examples, obtaining the sequencing reads includes sequencing the linear DNA molecules present in the sample. The sequencing can be a random sequencing of all the linear DNA molecules in the biological sample, rather than a targeted sequencing of particular molecules, and can be performed using any of the sequencing techniques described in Section II.A. In some instances, the biological sample is a cell-free biological sample and/or the linear DNA molecules are cell-free linear DNA molecules. For example, the biological sample can include or consist of plasma. The linear DNA molecules of the biological sample are naturally found in the biological sample in a linear form, and the provided method does not include operations, such as enzymatic cleavage or mechanical shearing, intended to linearize circular DNA molecules of the biological sample. In some instances, one obtained sequence read includes both the 5′ end sequence and the 3′ end sequence of a linear DNA molecule. In other instances, one obtained sequence read includes the 5′ end sequence of a linear DNA molecule, and another obtained sequence read includes the 3′ end sequence of the linear DNA molecule. The obtained sequence reads can each independently have a length that is at least 25 bp, at least 45 bp, at least 75 bp, at least 150 bp, at least 250 bp, at least 500 bp, at least 1 kb, at least 3 kb, at least 10 kb, at least 30 kb, or at least 100 kb

At block 520, the 5′ end sequence of the linear DNA molecule and the 3′ end sequence of the linear DNA molecule obtained in block 510 are mapped to a reference genome. Block 520 can be performed in a similar manner to block 420 of method 400. As with block 420, the mapping can be performed as described in Section II.A, and can involve independently determining an optimal alignment for each of the 5′ end sequence and the 3′ end sequence to the reference genome. In some instances, the alignments are each independently required to satisfy a mapping quality condition, for example, having a mapping quality that is at least 20, at least 25, at least 30, at least 35, at least 40, at least 45, or at least 50. In some cases, the mapping of the end sequences includes determining a genomic coordinate for each of the end sequences.

Additionally or alternatively, the mapping of the end sequences can include determining a genomic orientation for each of the end sequences. In some examples, the provided method also includes additionally mapping one or more sequences of the linear DNA molecule other than the 5′ end sequence and the 3′ end sequence, and determining the genomic coordinates and/or genomic orientations of these additionally mapped sequences. In some instances, the mapping further includes identifying if a sequence read includes a junction locus, and optionally determining the genomic coordinate of this junction.

At block 530, the linear DNA molecule is classified, based on the mapping in block 520, according to whether or not the linear DNA molecule was cleaved in vivo from a circular DNA molecule, i.e., whether or not the linear DNA molecule is an eccDNA remnant. Block 530 can be performed in a similar manner to block 430 of method 400. As with block 430, the classifying of the linear DNA molecule can be performed as described in Section II.B. For instance, the classifying can include comparing the genomic coordinate of the 5′ end sequence of the linear DNA molecule to the genomic coordinate of the 3′ end sequence of the linear DNA molecule. Additionally or alternatively, the classifying can include comparing the genomic orientation of the 5′ end sequence to the genomic orientation of the 3′ end sequence. As one example, the linear DNA molecule can be classified as an eccDNA remnant if (1) the genomic coordinate of the 5′ end sequence is larger than the genomic coordinate of the 3′ end sequence, and (2) the genomic orientation of the 5′ end sequence is identical to the genomic orientation of the 3′ end sequence. In some cases, the classifying can include comparisons of genomic coordinates and/or genomic orientations of mapped sequences other than or in addition to the 5′ end sequence and the 3′ end sequence.

At block 540, the members of a set all linear DNA molecules classified as being an eccDNA remnant in block 530 are analyzed to determine a property of the biological sample or the subject from whom the biological sample was obtained. In some instances, all members of the set of classified eccDNA remnants are analyzed. In other instances, a portion of the set of classified eccDNA remnants are analyzed. The analyses of the eccDNA remnants can be used in some examples to determine a collective value of the set of eccDNA remnants. One example of such a collective property is a count, e.g., an absolute count or a relative or normalized count, of eccDNA remnants in the set. Another example of such a collective property is a size distribution of eccDNA remnants in the set. Other examples of collective properties include a genomic distribution, a nucleotide motif frequency, and a methylation density.

The collective value can be used to determine the property of the sample or the subject. In some cases, a count of the set of eccDNA remnants is used to determine the property of the sample or subject, for example by comparing the count with a reference value and determining if the count is greater than, less than, or equal to the reference value. In other cases, a size distribution of the set of eccDNA remnants is used to determine the property of the sample, for example by determining a percentage of the set of eccDNA remnants that exceed a size threshold, e.g., a predetermined size threshold, and optionally then determining if this percentage is greater than, less than, or equal to a reference percentage value. In still other cases, the property of the sample is determined using a normalized genomic coverage of the eccDNA remnants, for example where the normalized genomic coverage is calculated by counting a subset of eccDNA fragments mapping to regions within a class of genomic elements, and dividing this count by the percentage of the genome withing the regions. In some cases, the property of the sample is determined using a frequency of one or more nucleotide motif patterns occurring for the set of eccDNA remnants, for example where at least one of the nucleotide motif patterns includes a plurality of nucleotide motifs, e.g., trinucleotide motifs, dinucleotide motifs, or tetranucleotide motifs. In other cases, the property of the sample is determined using a methylation status of eccDNA remnants having a size within a specified size range, for example, a size less than a maximum size (e.g., 1000 bp) or a size greater than a minimum size (e.g., 800 bp).

### IV. Example Properties of Sample or Subject

The provided methods for analyzing eccDNA remnants in a biological sample can be used to determine a variety of different useful properties of a biological sample, or of a subject from whom the biological sample was obtained. Because the methods use information related to eccDNA remnants that are detected, i.e., classified, from among other linear DNA molecules in the sample, and because previous methods did not use this information, the provided methods enable otherwise unavailable routes for analyzing a sample and/or evaluating a subject. For example, the methods can provide improved techniques for noninvasive medical or diagnostic assays.

### A. Pathology Classification

In some examples, the provided methods for analyzing eccDNA remnants in a biological sample can be used for recognizing, classifying, or treating pathologies of a subject. The analyzed eccDNA remnants can be from a biological sample obtained from a subject being screened for one or more particular pathologies. The inclusion of eccDNA remnants in the determination of a pathology classification can increase detection accuracy, for example because other pathology classifications do not use information related to eccDNA remnants. Furthermore, because a chromosome could repair itself after releasing an eccDNA, a chromosomal, i.e., genomic, copy of a sequence may no longer show a particular aberration associated with a pathology, whereas the corresponding eccDNA remnant may.

The classification of a pathology determined by the provided methods can include the level of disease such as cancer, e.g., where the subject is being screened for cancer. The level of disease can be of a particular organ, e.g., the liver. For instance, pathology classifications can include a level of disease of the liver, where the level can be determined to be, for example, cancer, a hepatitis B virus (HBV) infection, or no disease. In another example, the level of disease includes an indication of whether a transplanted organ is being rejected.

Determining the level of a disease can include, for example, comparing a collective value (e.g., a count or a size distribution) of the classified eccDNA remnants to a reference value, and determining the level of disease based on the comparison. The reference value can be determined based on cohorts of subjects that have a known level of the disease. The reference value (e.g., a cutoff value) can be selected to optimize a specificity and sensitivity to predicting the level of disease. Thus, the reference value can be determining using a training set of samples from subjects that all have the disease, do not have the disease, or a combination of both. Accordingly, the reference value can be determined based on reference separation values determined from samples of subjects having a known level of disease.

In some instances, the provided methods can further include treating the subject for the disease or condition indicated by a pathology classification, thereby improving the disease or condition (e.g., by its removal or by a reduction in its severity). Treatments can be selected and/or provided according to a determined level of the disease or disorder, the identified variants, and/or the tissue of origin. For example, an identified variant can be targeted with a particular drug or chemotherapy. Additionally or alternatively, the level of a disorder as indicated by a pathology classification can be used to determine how aggressive of a treatment should be used.

Machine learning models can also be used with the provided methods to, for example, determine a level of a disease. Exemplary models include, but are not limited to, those using linear regression, logistic regression, neural networks such as deep recurrent neural network, Bayes classifier, hidden Markov model (HMM), linear discriminant analysis (LDA), k-means clustering, density-based spatial clustering of applications with noise (DBSCAN), decision tree (e.g., random forest), and support vector machine (SVM). The model can include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm.

### B. Fractional DNA Concentration

In some examples, the provided methods for analyzing eccDNA remnants in a biological sample can be used for determining the fraction of certain DNA of interest in a biological sample. The DNA of interest can be, for example, clinically relevant DNA. The DNA of interest can be DNA from one or more particular tissues. In some examples, the provided methods are useful for determining the fraction of DNA from a tumor tissue present in a biological sample. In other examples, the provided methods are useful for determining the fraction of DNA from a transplanted tissue present in a biological sample. In still other examples, the provided methods are useful for determining the fraction of DNA from fetal tissue present in a biological sample. Because previous approaches for determining fractional DNA concentrations did not consider or analyze eccDNA remnants present in a biological sample, the methods disclosed herein advantageously provide a new source of information that can be useful in providing more accurate assessments of fractional DNA concentrations through noninvasive sampling and assays.

In some examples, determining a fractional DNA concentration involves comparing a reference value to a collective value (e.g., a count or a size distribution) of the classified eccDNA remnants in a biological sample from the subject. The reference value can be determined based on the level of the collective value as measured in one or more other subjects, or the same subject, having one or more known fractional DNA concentrations. The reference value (e.g., a cutoff value) can be selected to optimize a specificity and/or sensitivity to estimating a fractional DNA concentration. Thus, the reference value can be determining using a training set of samples taken from subjects having different fractional DNA concentrations. For example, the reference value can be determined based on reference separation values determined from samples of subjects having different known fractional DNA concentrations.

### V. Methods for Detecting Cancer

In some aspects, the present disclosure provides various methods for determining a cancer level of a subject, where the cancer level is determined by analyzing eccDNA remnants in a biological sample from the subject as described in Section IV.A. Because previous approaches for determining a cancer level of a subject did not consider or analyze eccDNA remnants present in a biological sample, the methods disclosed herein advantageously provide a new source of information that can be useful in providing more accurate assessments of cancer levels through noninvasive sampling and assays.

Biological samples can be obtained from a subject (e.g., a subject having or suspected of having a cancer) at various time points and analyzed independently at those time points. In some examples, samples are obtained from a subject at time points before and after a treatment of cancer (e.g. targeted therapy, immunotherapy, chemotherapy, surgery). In other examples, samples are obtained from a subject at time points before and after a diagnosis of cancer. In further examples, samples are obtained from a subject at time points before and after a progression of cancer, before and after a development of metastasis, before and after an increased severity of disease, and/or before and after development of complications.

In some instances, the methods further include providing a treatment appropriate for the level of cancer determined in the subject. Various treatments can be performed. A treatment can include any suitable therapy, including with a drug, chemotherapy, radiation, immunotherapy, hormone therapy, stem cell transplant, surgery, or other suitable cancer treatment. The cancer treatment can be targeted, e.g., using precision medicine tailored to the specific properties of the disease, e.g., a particular genetic composition of a tumor. Based on the determined level of the cancer, a treatment plan can be developed to decrease the risk of harm to the subject. Methods can include treating the subject according to the treatment plan.

A. Results Using eccDNA Remnant Counts and Size Distributions

As an example, the provided methods for determining a cancer level were used to analyze biological samples from subjects who were either HBV carriers or hepatocellular carcinoma (HCC) patients. The analytical procedures described in the foregoing sections were used to detect and classify eccDNA remnants in the biological samples using sequence reads obtained using linear DNA sequencing protocols. Specifically, previously generated nanopore sequencing data obtained from patients having different cancer levels were analyzed according to aspects of the provided methods, and the analyses successfully identified different eccDNA remnant properties (e.g., differences in count and size distribution) that related to the different cancer levels of these subjects.

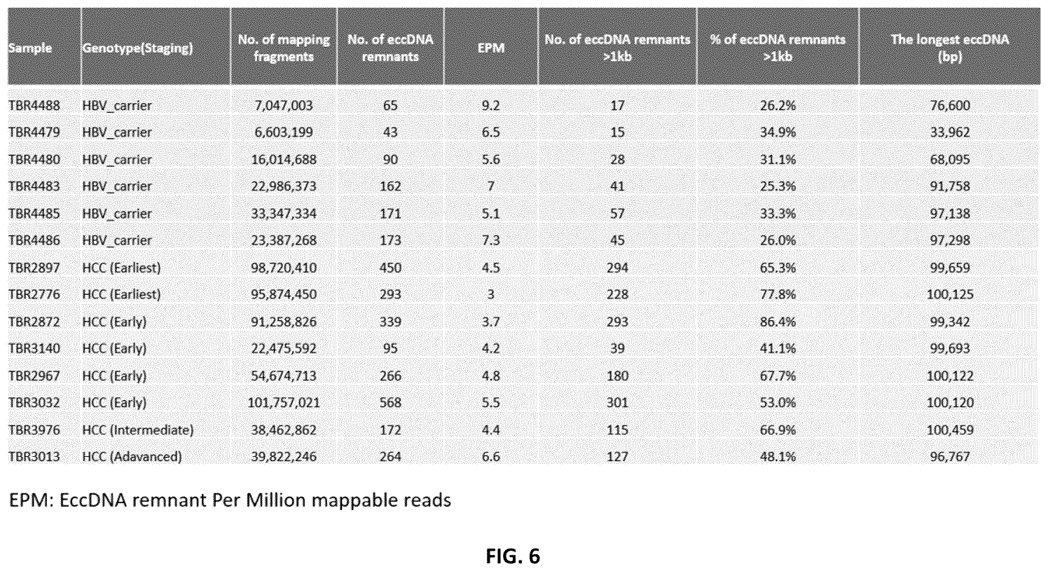

FIG. 6 shows nanopore sequencing data used in this example based on biological samples from six subjects who were carriers of HBV, and eight subjects having HCC. A median number of 19,500,531 (range: 6,603,199-33,347,334) sequencing read counts, i.e., mapping fragments, were received from the HBV subjects, and a median of 72,966,770 (range: 22,475,592-101,757,021) sequencing read counts were received from the HCC patients. Using the methods described in Sections II.C and III.F, a median of 126 (range: 43-173) eccDNA remnants were classified in each sample from the HBV subjects based on the mapping of the sequence reads. And a median of 280 (range: 95-568) eccDNA remnants were similarly classified in each sample from the HCC subjects.

As also shown in FIG. 6, the raw counts of eccDNA remnants determined based on the sequencing reads from each of the 14 biological samples were normalized with respect to the count of mappable reads used in these determinations. The resulting relative counts are reported in FIG. 6 in terms of eccDNA remnants per million mappable reads (EPM), and show that between 5 and 10 EPM were classified, i.e., detected, in the HBV carrier samples, whereas between 2 and 7 EPM were classified in the HCC patient samples.

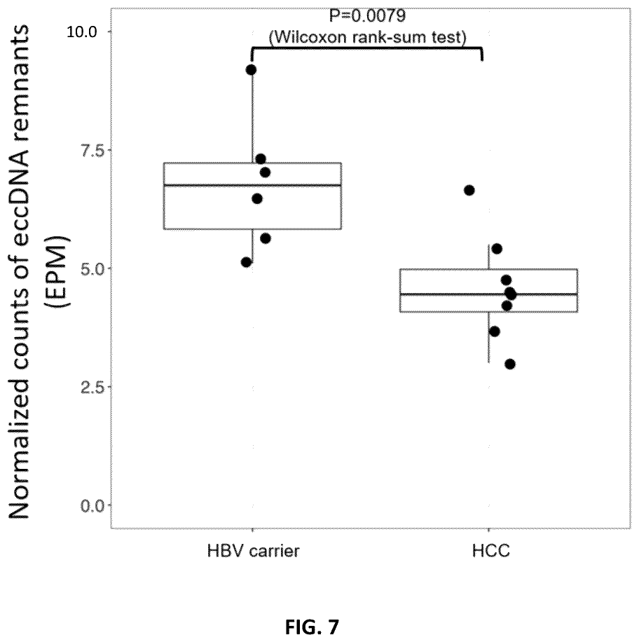

FIG. 7 is a graph plotting the data from the table of FIG. 6. The vertical y-axis of the graph represents the normalized count of eccDNA remnants per million mappable reads (EPM), and the data points indicate counts for different HBV carrier samples (left) and HCC patient samples (right). The plotted data show that relative eccDNA remnant counts are significantly lower in the HCC group compared to the HBV carrier group (P=0.0079, Wilcoxon rank-sum test).

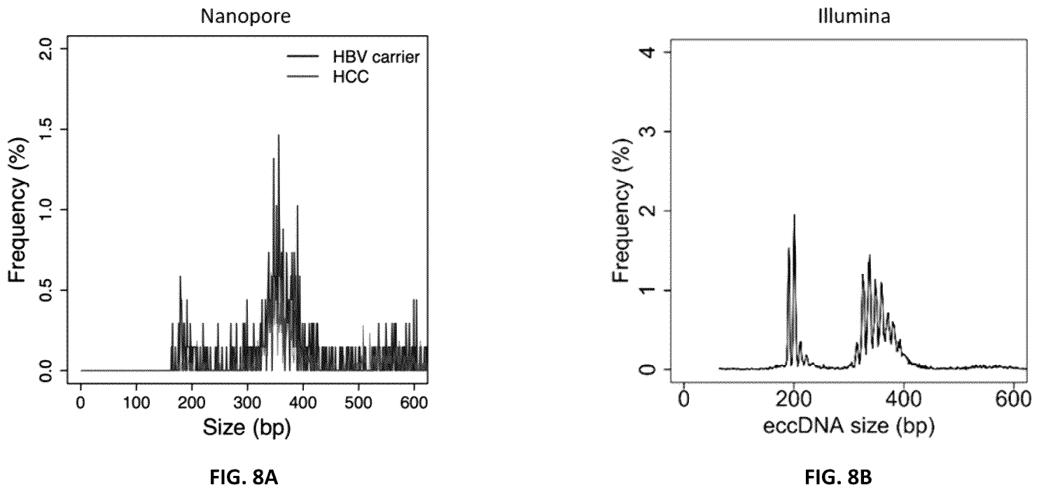

The sizes and size distributions of the eccDNA remnants in each of the 14 biological samples were also determined as described in Section III.B. As part of this analysis, the determined size profile of small eccDNA remnants in this experiment was compared with Illumina data for small eccDNA size profiling. The graphs of FIGS. 8A and 8B show that the size profiles determined with these two methods agreed well with one another, with both identifying two major peaks within this range at 200 bp and 350 bp.

Referring again to FIG. 6, the determined eccDNA remnant size distributions for the 14 biological samples were used to calculate the percentage of eccDNA remnants larger than 1 kb in each sample.

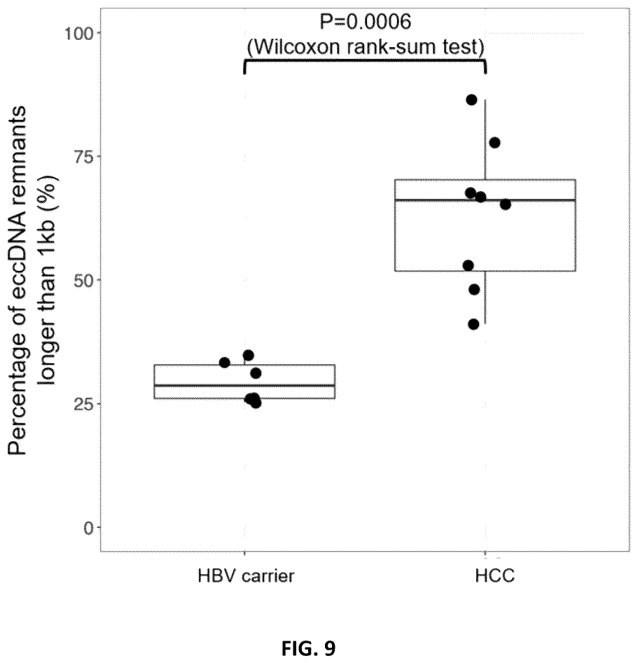

FIG. 9 presents this calculated data in graphical form, comparing the percentage of these large eccDNA between HBV carriers and HCC patients. The vertical y-axis of the graph represents the percentage of eccDNA remnants having a length longer than 1 kb, and the data points indicate percentage values for different HBV carrier samples (left) and HCC patient samples (right). The plotted data show a complete separation (P=0.0006, Wilcoxon rank-sum test). between the these groups in terms of percentages of eccDNA remnants larger than 1 kb.

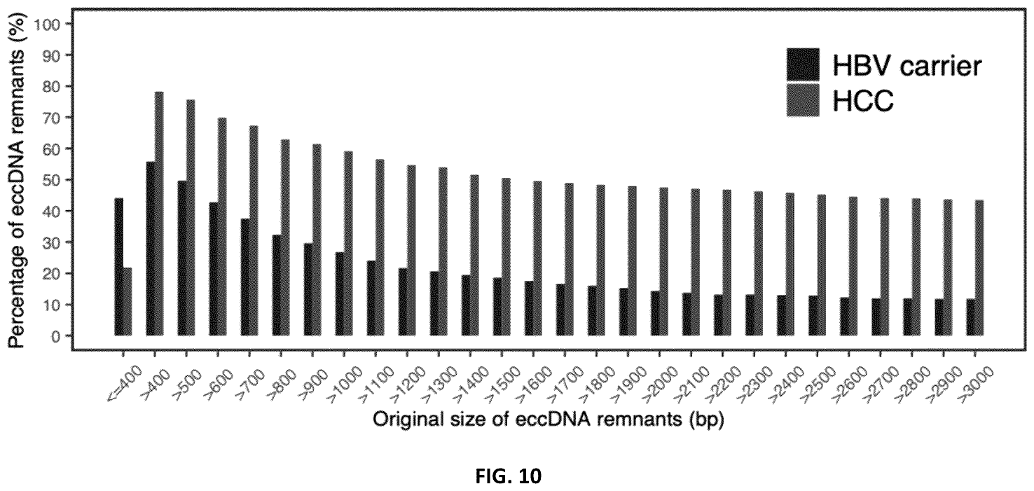

The graph of FIG. 10 plots the size distributions of eccDNA remnants classified in the HBV carrier biological samples and the HCC patient biological samples, where the distributions are shown for sizes ranging from smaller than 400 bp to larger than 3 kb. While the significant majority of the eccDNA remnants classified in the HCC patient samples were larger than 400 kb, nearly half of the eccDNA remnants classified in the HBV carrier samples were smaller than 400 kb. More specifically, 55.9%, 26.8%, 14.4%, and 11.9% of eccDNA remnants in the HBV carrier samples had sizes greater than 400 bp, 1 kb, 2 kb and 3 kb, respectively. In contrast, 78.3%, 59.2%, 47.5%, and 43.5% of eccDNA remnants in the HCC patient samples had sizes greater than 400 bp, 1 kb, 2 kb and 3 kb, respectively.

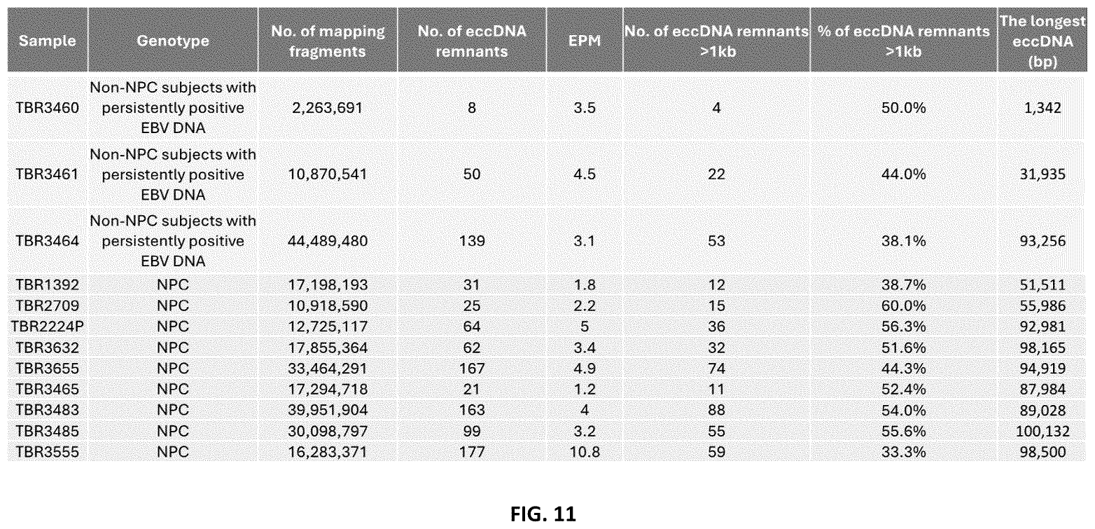

FIG. 11 presents nanopore sequencing data from another example of detecting or classifying a cancer using eccDNA remnant size distributions. The table of FIG. 11 includes data based on biological samples from three non-nasopharyngeal carcinoma (NPC) subjects displaying persistently positive EBV DNA levels, as well as from nine NPC patients. Using the methods described in Sections II.C and III.F, eccDNA remnants were classified in each of these twelve samples based on the mapping of the sequence reads. Additionally, the sizes and size distributions of the eccDNA remnants in each of the samples were determined as described in Section III.B.

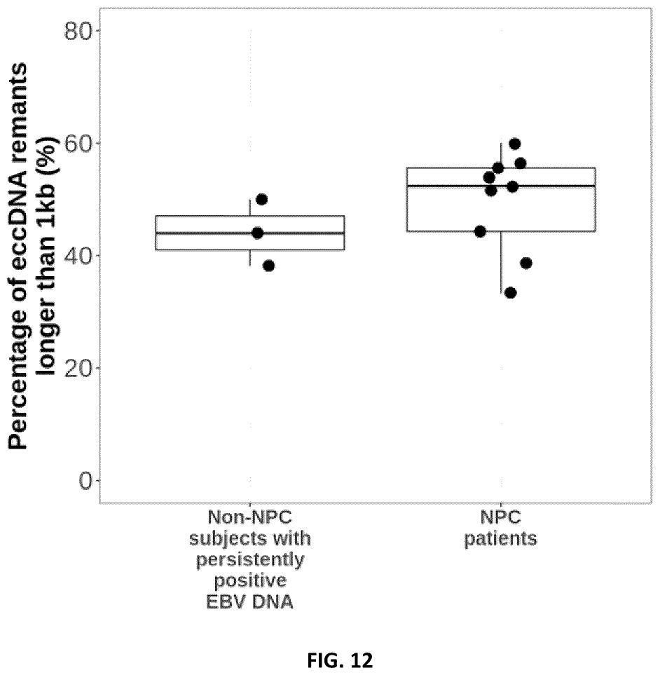

FIG. 12 provides a graph plotting data from the table of FIG. 11, comparing the frequency of large eccDNA remnants between non-NPC subjects and NPC patients. The vertical y-axis of the graph represents the percentage of eccDNA remnants having a length longer than 1 kb, and the data points indicate percentage values for different non-NPC subject samples (left) and NPC patient samples (right). As shown in FIG. 12, the median proportions of eccDNA remnants exceeding 1 kb in length were 44.0% for non-NPC subjects and 52.4% for NPC patients, respectively. Thus, there was an 8% elevation in NPC cases compared to non-NPC subjects. Together with the results from the HCC example described above and illustrated in FIGS. 6-10, these findings demonstrate that the size distributions and/or counts of eccDNA remnants can provide information for detecting different cancer types, where this detection can have important diagnostic implications.

### B. Results Using Genomic Distribution

In another example, the overall population of plasma eccDNA remnants identified for the HBV carrier and HCC patients from Section V.A was mapped to various classes of genomic elements. These classes encompass a range of genomic features, including but not limited to 5′ UTR, 3′ UTR, exon, intron, 2 kb upstream of genes (Gene2kbU), 2 kb downstream of genes (Gene2kbD), CpG island, 2 kb upstream of CpG Island (CGI2kbU), 2 kb downstream of CpG Islands, Alu, and others. The distribution of eccDNA remnants across these classes was quantified using a metric termed “normalized genomic coverage,” which was calculated as the percentage of eccDNA mapped to that class of genomic element divided by the percentage of genome covered by that particular class.

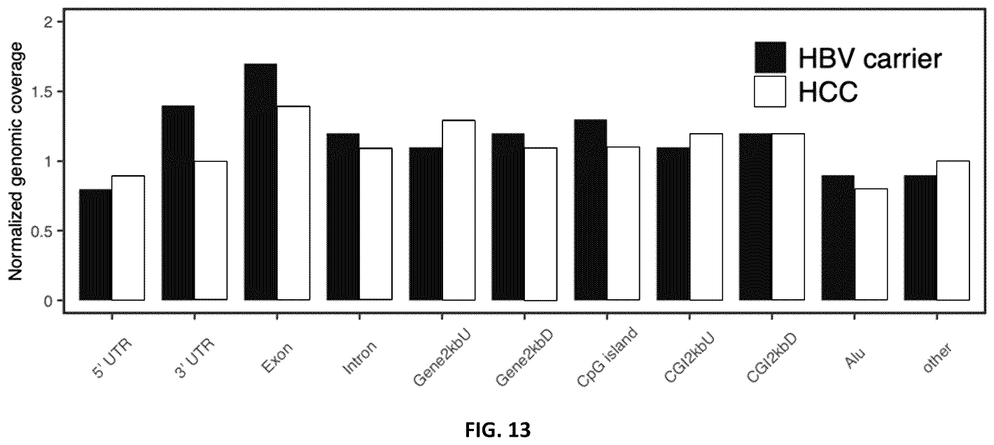

FIG. 13 is a graph comparing normalized genomic coverage between HBV carriers and HCC patients in different classes. The vertical y-axis of the graph represents the normalized genomic coverage of eccDNA remnants, and the horizontal x-axis lists several different classes of genomic elements or regions. For each of these different classes, the normalized genomic coverage for HBV carrier samples (left) and HCC patient samples (right) are plotted. Notably, for the 3′ UTR region, the normalized genomic coverage was 1.38 for HBV carriers and 1.00 for HCC patients, indicating a significant difference between the two types of eccDNA remnant samples. Additionally, for the Exon region, the normalized genomic coverage of 1.67 for HBV carriers was also significantly higher than the normalized genomic coverage of 1.42 for HCC patients. A contrasting pattern was observed in the Gene2kbU class, where the normalized genomic coverage was 1.06 for HBV carriers and 1.26 for HCC patients. This observation shows that among the different classes for which the normalized genomic coverages are significantly different between the HCC patients and the HBV carriers, for some classes a higher normalized genomic coverage can indicate cancer detection, while for other classes a lower normalized genomic coverage can indicate cancer detection.

These results demonstrate that analysis of eccDNA remnants to determine their normalized genomic coverage can provide useful information for detecting cancer. For example, a comparison of normalized genomic coverage across different classes can yield insights into potential genomic variations between HBV carriers and HCC patients, which can help uncover specific genes or regulatory mechanisms that play a role in disease progression.

### C. Results Using Nucleotide Sequence Motifs

In other examples, nucleotide motifs and motif patterns flanking the junction sites of eccDNA remnants were investigated, where the motifs and patterns indicate the locations where these fragments were excised from the genome prior to eccDNA formation. For these nonlimiting examples, analyzed DNA sequences included those within a range of 50 base pairs upstream and downstream of the start and end positions of eccDNA remnant junction sites.

FIG. 14 illustrates a section of DNA (e.g., genomic DNA) from which an eccDNA fragment was excised. The Start and End genomic positions of the excised eccDNA are indicated in FIG. 14. Also shown in the illustration are three trinucleotide segments identified as I, II, III, and IV, where segments I and II are on opposite sides of the Start genomic position, and segments III and IV are on opposite sides of the End genomic position. Segments I and II are separated by spacer region S1, where S1 includes the eccDNA Start genomic position. And segments III and IV are separated by spacer region S2, where S2 includes the eccDNA End genomic position. In the particular example illustrated in FIG. 14, S1 and S2 each have lengths of 4 bp. Furthermore, spacer region S1 is located such that the nucleotide of segment I that is closest to the Start position is 3 bp away from the Start position, and the nucleotide of segment II that is closest to the Start position is 2 bp away from the Start position. Similarly, spacer region S2 is located such that the nucleotide of segment IV that is closest to the End position is 3 bp away from the End position, and the nucleotide of segment III that is closest to the End position is 2 bp away from the End position.

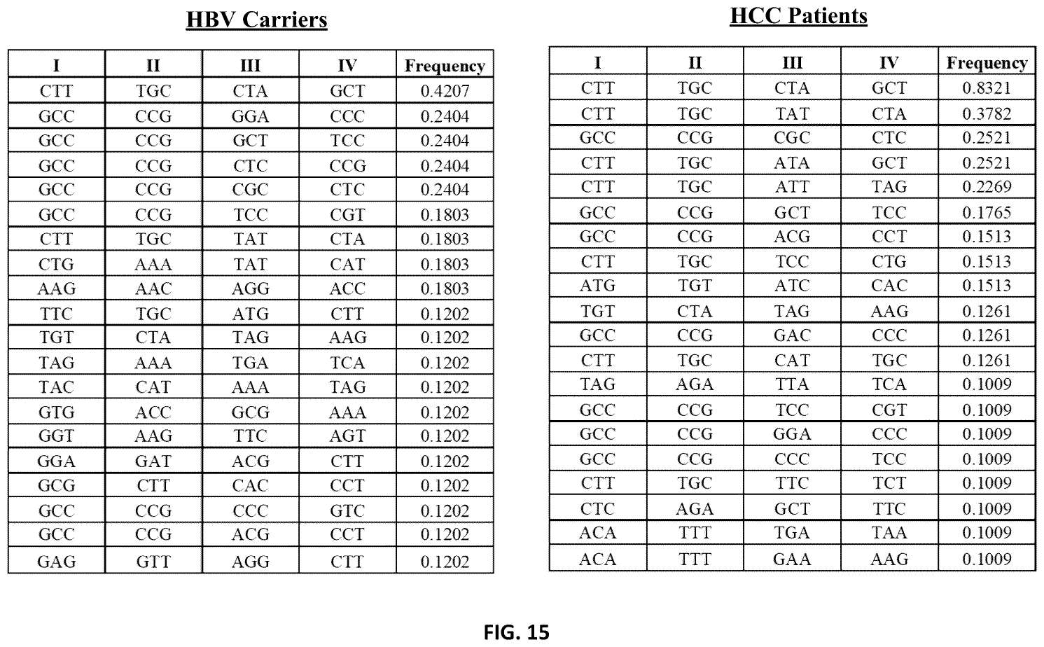

FIG. 15 presents a pair of tables listing results from one example analyzing segments I, II, III, and IV in the specific positions illustrated in FIG. 14. In this analysis, the sequences of each of the four trinucleotide segments (I, II, III, and IV) were compared between HBV carriers (left table) and HCC patients (right table). The left and right tables list the 20 most frequently occurring combinations of these trinucleotide segments in HBV carriers and HCC patients, respectively. For instance, the combination of “CTT” (segment I), “TGC” (segment II), “CTA” (segment III), and “GCT” (segment IV) occurred with a frequency of 0.4207 in HBV carriers, whereas in HCC patients, the frequency of this combination increased significantly to 0.8321. This substantial increase in frequency indicates a notable association with HCC. Similarly, for the combination “CTT,” “TGC,” “TAT,” and “CTA,” the frequency rose from 0.1803 in HBV carriers to 0.3782 in HCC patients. This approximately twofold overrepresentation suggests that the presence of these combinations of motif patterns can serve as a key feature in disease progression and serve as a biomarker for cancer diagnosis.

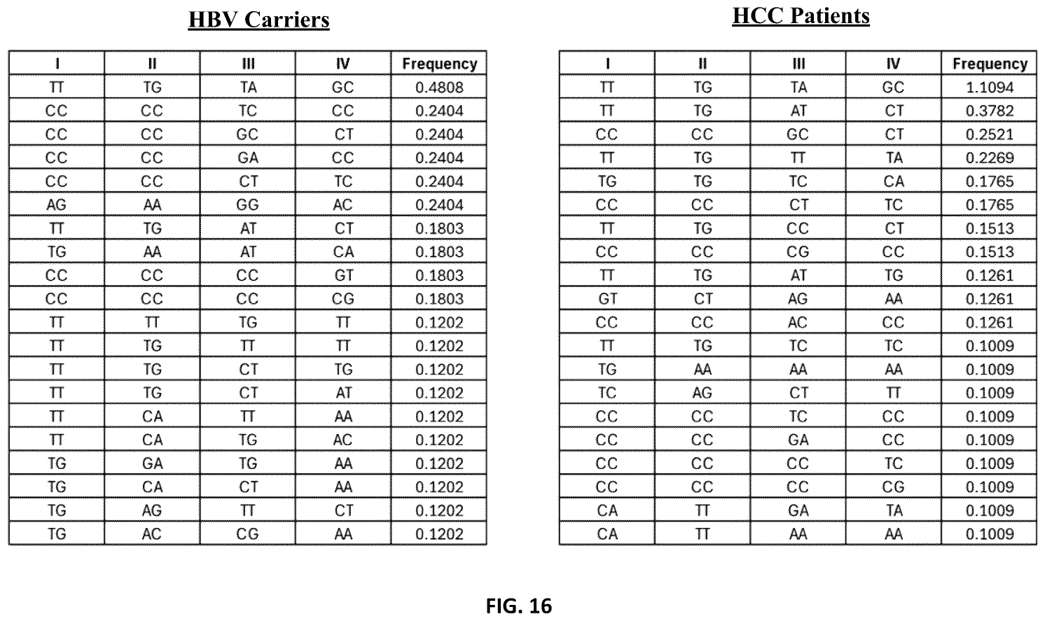

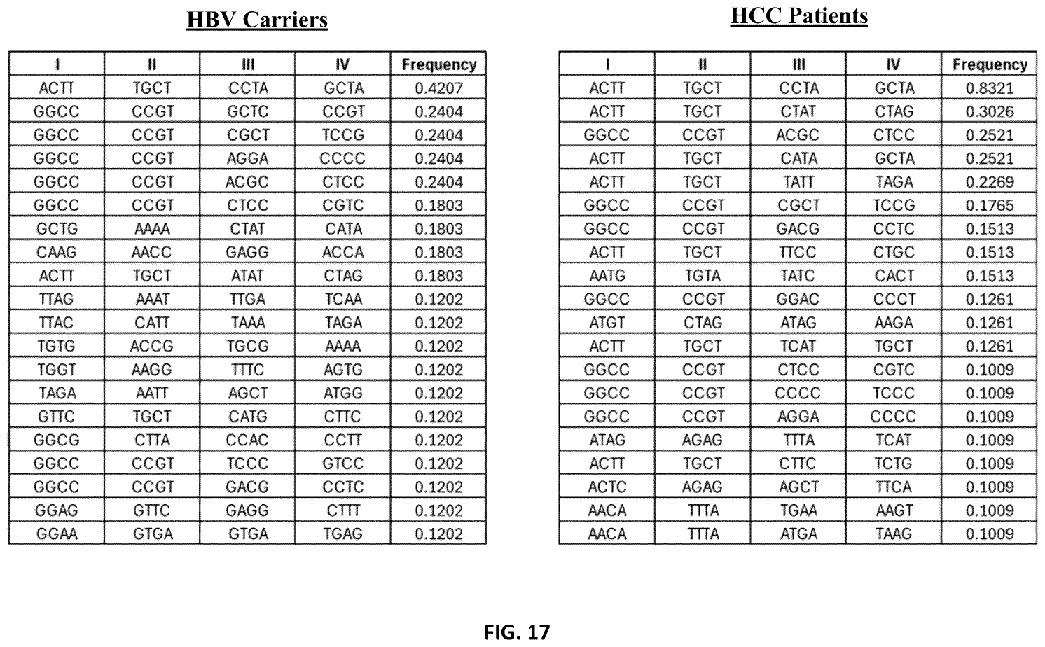

FIGS. 16 and 17 each present a pair of tables listing results from other examples analyzing patterns of four sequence motifs positioned about the eccDNA genomic Start and End positions as in FIGS. 14 and 15, but having motif lengths of 2 nt and 4 nt instead of 3 bp. Specifically, the data of FIG. 16 was generated by analyzing four dinucleotide segments (I, II, III, and IV). In this case, the nucleotide of dinucleotide segment I that is closest to the Start position is 3 bp upstream from the Start position, and the nucleotide of dinucleotide segment II that is closest to the Start position is 2 bp downstream from the Start position. The nucleotide of dinucleotide segment IV that is closest to the End position is 3 bp downstream from the End position, and the nucleotide of dinucleotide segment III that is closest to the End position is 2 bp upstream from the End position. To generate the data of FIG. 17, four tetranucleotide segments (I, II, III, and IV) were analyzed. In this case, the nucleotide of tetranucleotide segment I that is closest to the Start position is 3 bp upstream from the Start position, and the nucleotide of tetranucleotide segment II that is closest to the Start position is 2 bp downstream from the Start position. The nucleotide of tetranucleotide segment IV that is closest to the End position is 3 bp downstream from the End position, and the nucleotide of tetranucleotide segment III that is closest to the End position is 2 bp upstream from the End position.

The data in FIGS. 16 and 17 show that combinations of dinucleotide motif patterns or tetranucleotide motif patterns also can be used to differentiate cancer samples from non-cancer samples. For example, FIG. 16 indicates that the combination of “TT” (segment I), “TG” (segment II), “TA” (segment III), and “GC” (segment IV) occurred with a frequency of 0.4808 in HBV carriers, whereas in HCC patients, the frequency of this combination increased significantly to 1.1094. Similarly, FIG. 17 indicates that for the combination of “ACTT” (segment I), “TGCT” (segment II), “CCTA” (segment III), and “GCTA” (segment IV), the frequency rose from 0.4207 in HBV carriers to 0.8321 in HCC patients. These results demonstrate that the size of the analyzed motifs can be varied without losing the ability of the analysis to detect a cancer.

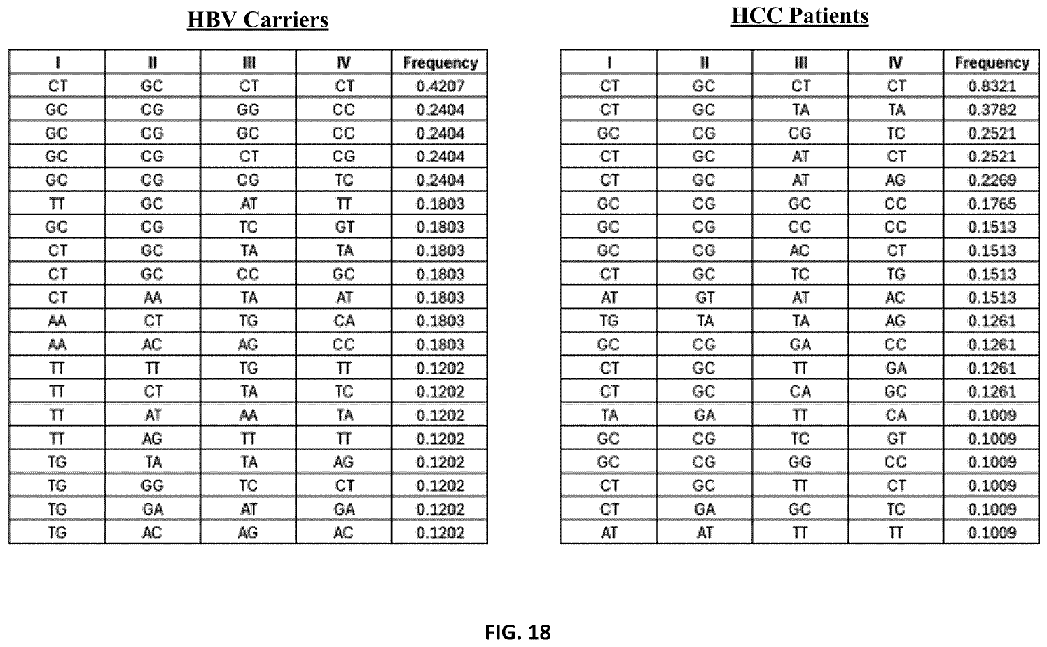

FIG. 18 presents a pair of tables listing results from an example analyzing patterns of dinucleotide segments (I, II, III, and IV) located in different positions than the dinucleotide segments of FIG. 16. The data of FIG. 18 was generated by analyzing a dinucleotide segment I for which the nucleotide closest to the Start position is 4 bp upstream of the Start position, a dinucleotide segment II for which the nucleotide closest to the Start position is 3 bp downstream of the Start position, a dinucleotide segment III for which the nucleotide closest to the End position is 3 bp upstream of the End position, and a dinucleotide segment IV for which the nucleotide closest to the End position is 4 bp downstream of the End position. The data in FIG. 18 shows that the positions of the analyzed motifs can be varied without losing the ability of the analysis to detect cancer. For instance, FIG. 18 indicates that the combination of “CT” (segment I), “GC” (segment II), “CT” (segment III), and “CT” (segment IV) occurred with a frequency of 0.4207 in HBV carriers, whereas in HCC patients, the frequency of this combination increased to 0.8321.

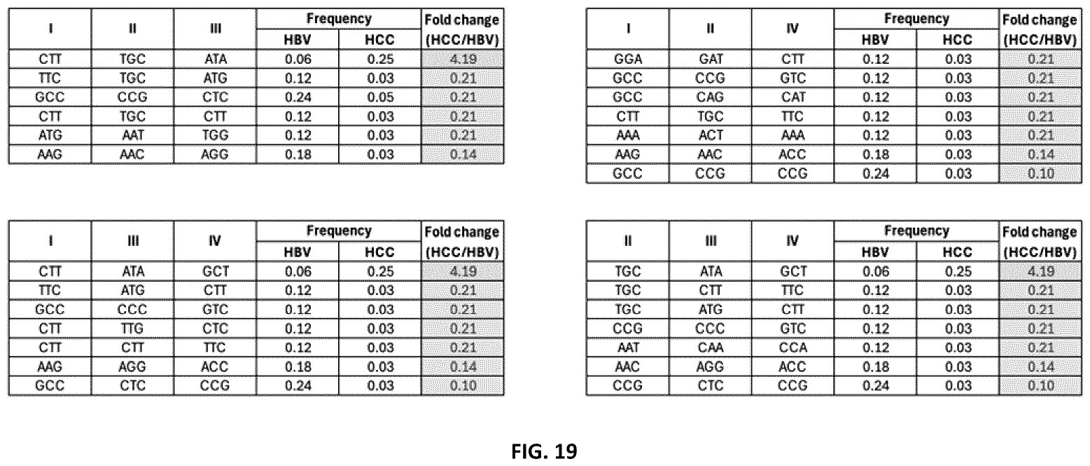

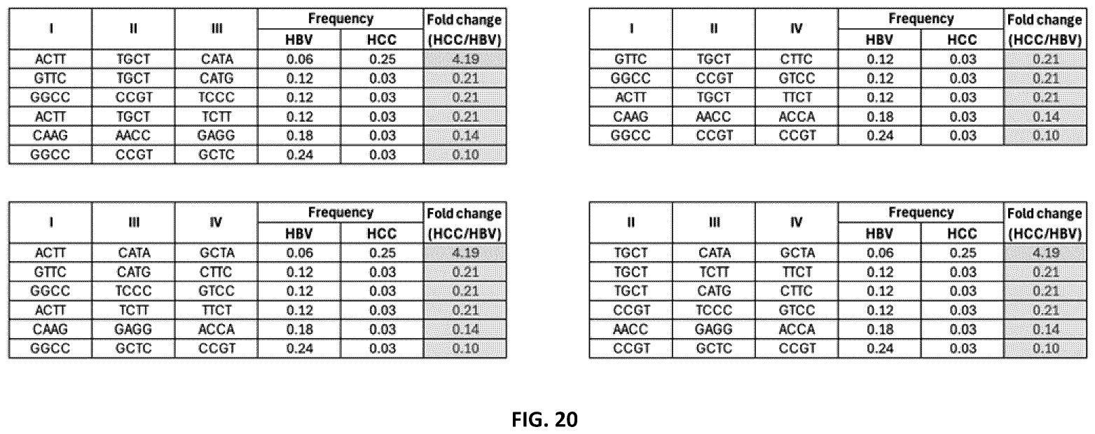

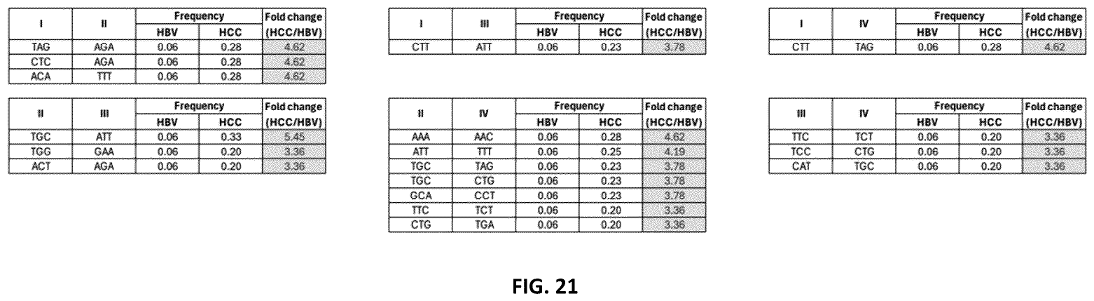

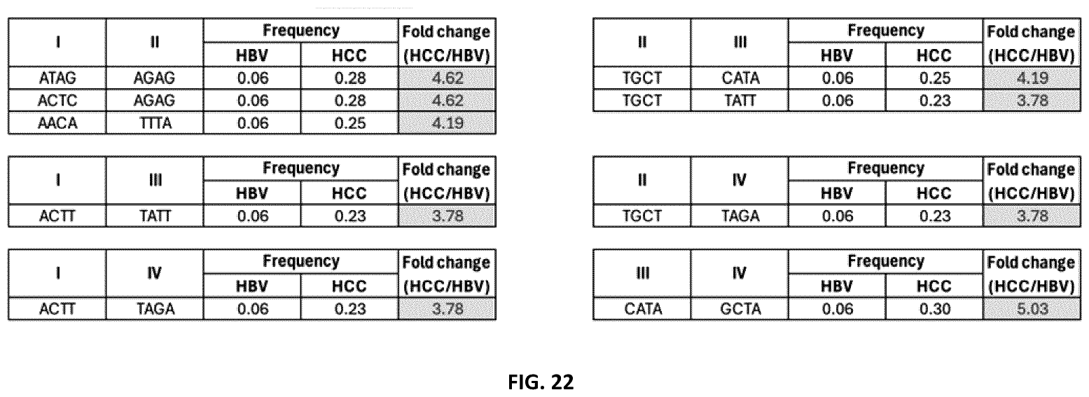

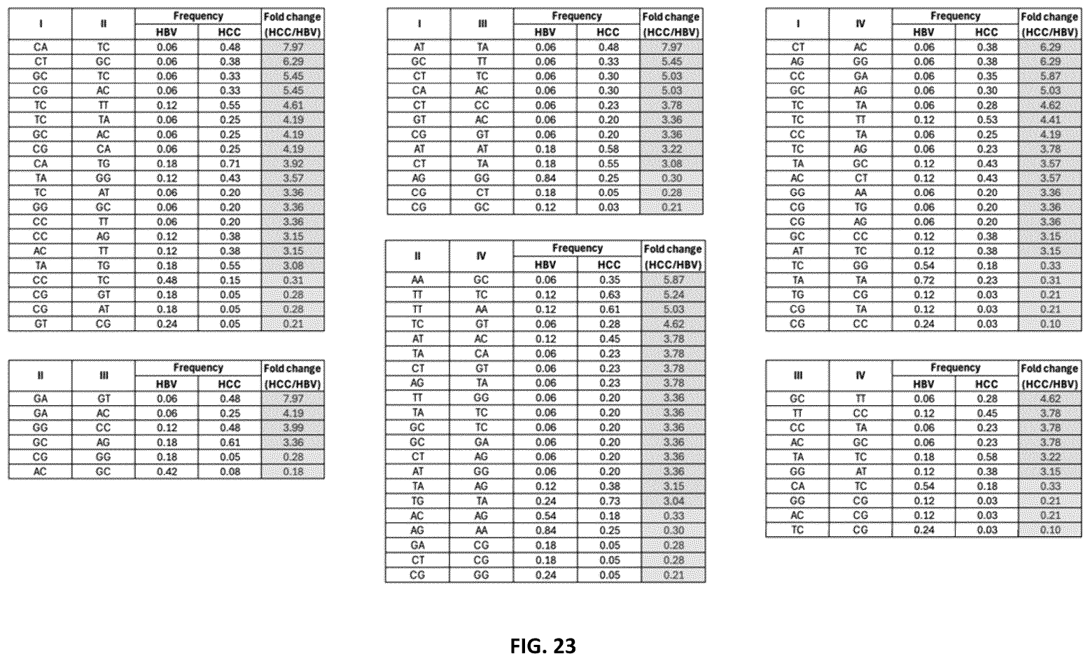

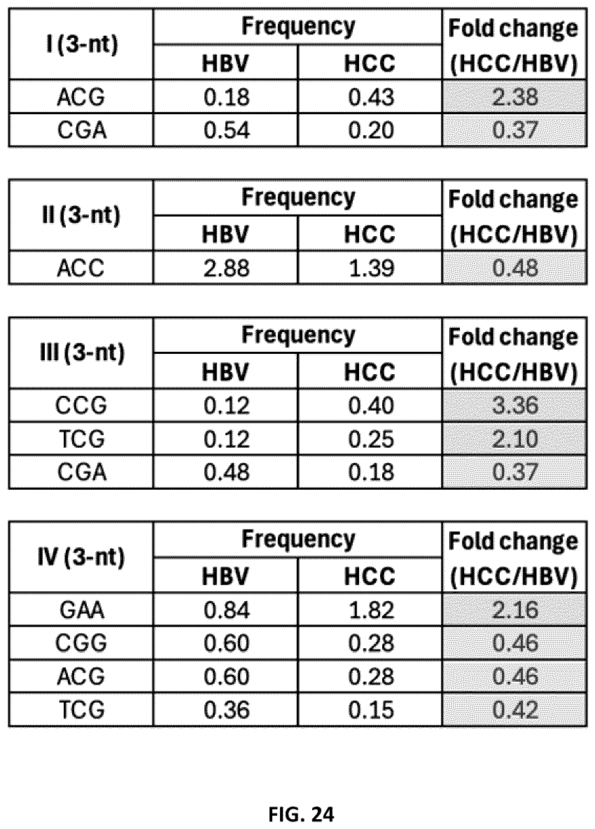

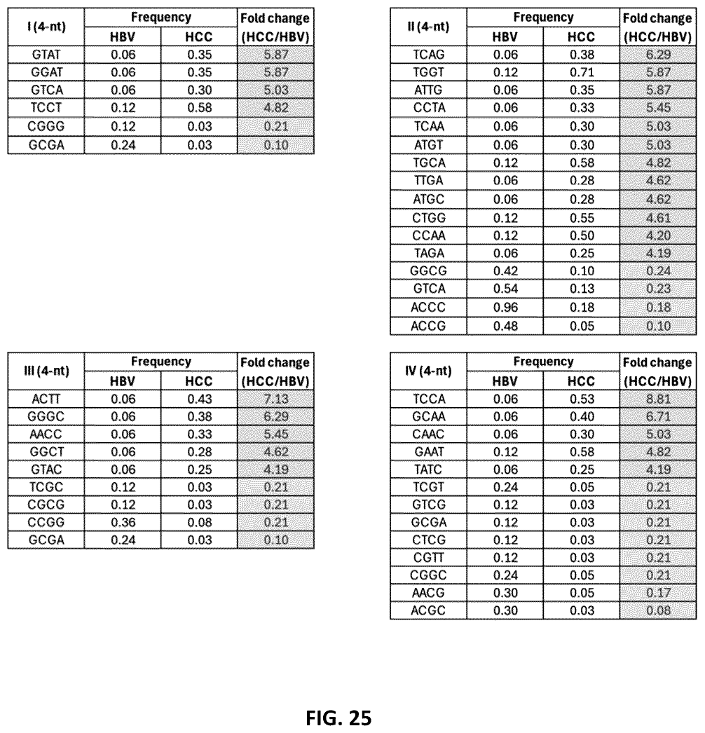

FIGS. 19-25 present tables listing results from additional examples analyzing patterns that each consist of fewer than four motif segments. While the trinucleotide (FIGS. 19, 21, and 24), tetranucleotide (FIGS. 20, 22, and 25) and dinucleotide (FIG. 23) motif segments analyzed in these additional examples are identical to ones described previously in FIGS. 15, 17, and 16, respectively, the analyses of these motif segments considered three-motif patterns (FIGS. 19 and 20), two-motif patterns (FIGS. 21-23), and singular motif segments. (FIGS. 24 and 25). In FIG. 19, the data shows that analyses of patterns of three of the trinucleotide segments (I, II, and III; I, II, and IV; I, III, and IV; and II, III, and IV) are sufficient to identify at least 4-fold differences between frequencies in HBV and HCC samples. Similarly, the data of FIG. 20 shows that analyses of patterns of three of the tetranucleotide segments can also identify at least 4-fold differences between HBV and HCC. Results from other examples confirmed that at least 3-fold differences between the HBV and HCC samples could be detected using patterns of two of the trinucleotide (FIG. 21), tetranucleotide (FIG. 22), or dinucleotide (FIG. 23) motif segments. And even using only a single motif segment according to the provided methods enabled discrimination between the cancer and non-cancer samples (FIGS. 24 and 25).

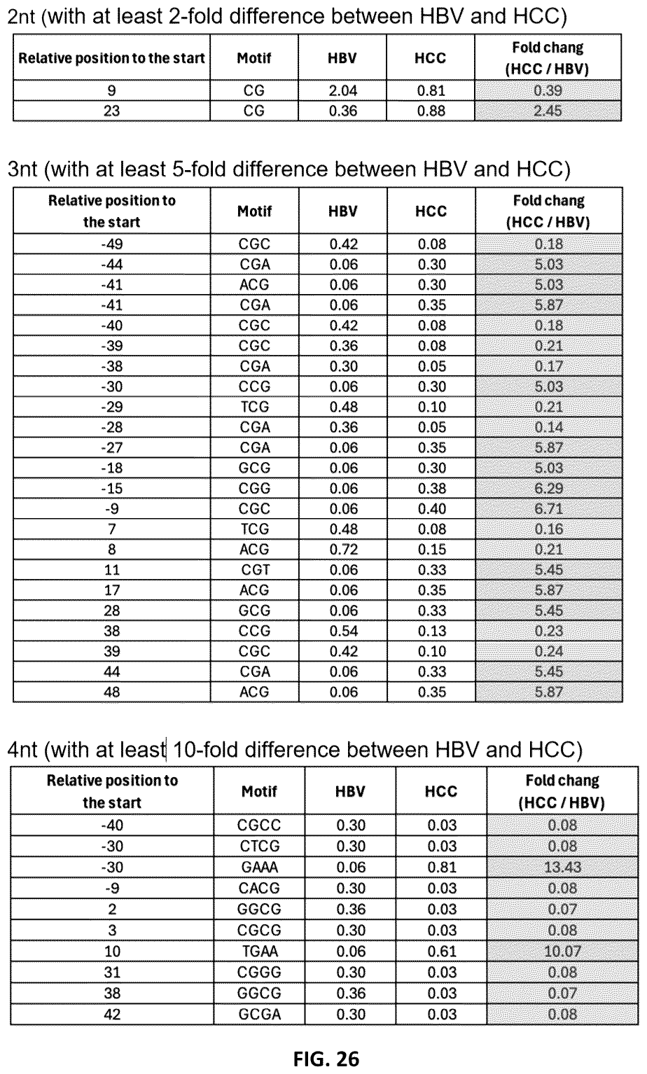

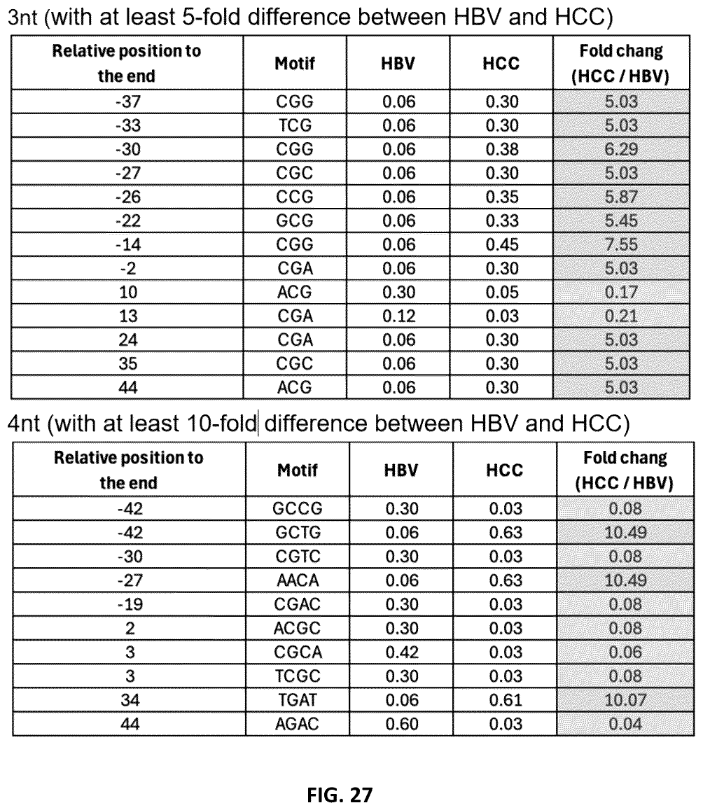

FIGS. 26 and 27 provide data from another example confirming that analysis of a single motif segment proximate to eccDNA remnant junction sites can detect a cancer state of a biological sample or subject. In this example, the single motif segments that were analyzed were not first identified as being one segment of a larger pattern of multiple segments, as in the examples of FIGS. 15-25. Instead, the segments analyzed in this example included single dinucleotide, trinucleotide, or tetranucleotide motifs located within a specified distance (e.g., 50 bp) upstream or downstream from the Start position (FIG. 26) or End position (FIG. 27) of the eccDNA remnants in the genome. The data presented in the tables of FIGS. 26 and 27 demonstrate that significant frequency differences that were at least as high as 10-fold could be observed using this provided approach.

### D. Results Using Methylation Density

In other examples, the methylation densities of eccDNA fragments within varying size ranges were investigated. Nanopore sequencing enables direct, real-time sequencing and detection of DNA base modifications, including but not limited to 5mC, 5hmC, 6 mA, and/or 4mC, without the need for additional chemical conversion or experimental preparation, distinguishing it from methods like bisulfite sequencing. Leveraging nanopore sequencing data, the methylation features of eccDNA remnants identified in plasma from HBV carriers and HCC patients can be delineated. The overall methylated CpG density of these eccDNA remnants (MDᵣₑₘₙₐₙₜ) is calculated by the equation:

$${MD}_{remnant} = \frac{M}{M + U}$$

where M represents the count of methylated CpG sites across all eccDNA remnants and U denotes the count of unmethylated CpG sites within the same plasma sample.

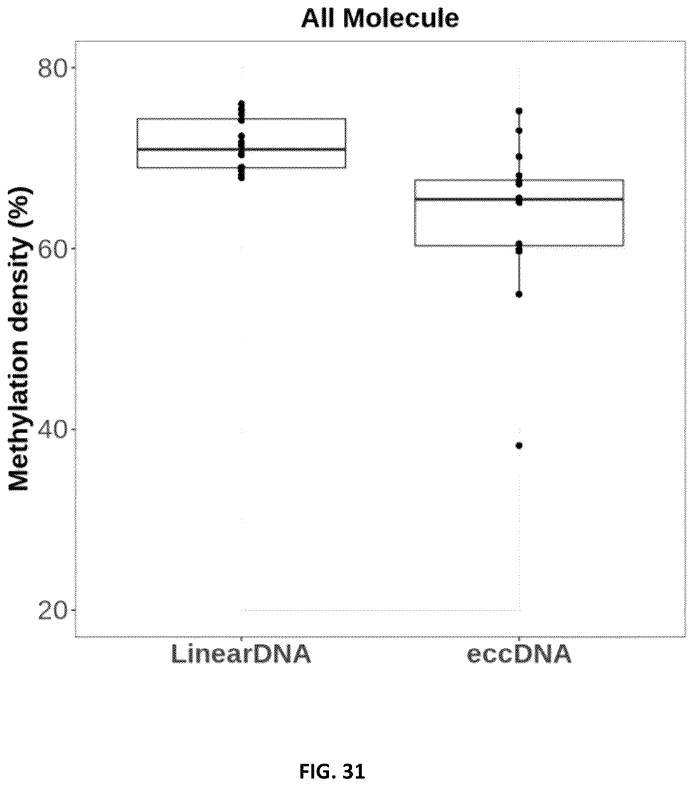

FIG. 31 shows a graph comparing methylation density values for linear DNA and eccDNA remnants. The vertical y-axis of the graph represents the methylation density percentage, and the data points indicate percentage values for linear DNA (left) and eccDNA remnants (right) identified in biological samples from HBV carriers. As shown in FIG. 31, the median methylation densities for eccDNA remnants differed significantly from that of linear DNA (Wilcocoxon rank sum test, P-value=0.00008). This difference demonstrates that methylation of eccDNA is not entirely similar to that of conventional linear cell-free DNA. As a result, behaviors observed for linear DNA methylation (e.g., differences between linear DNA methylation from diseased and non-diseased samples) are different from behaviors for eccDNA remnant methylation.

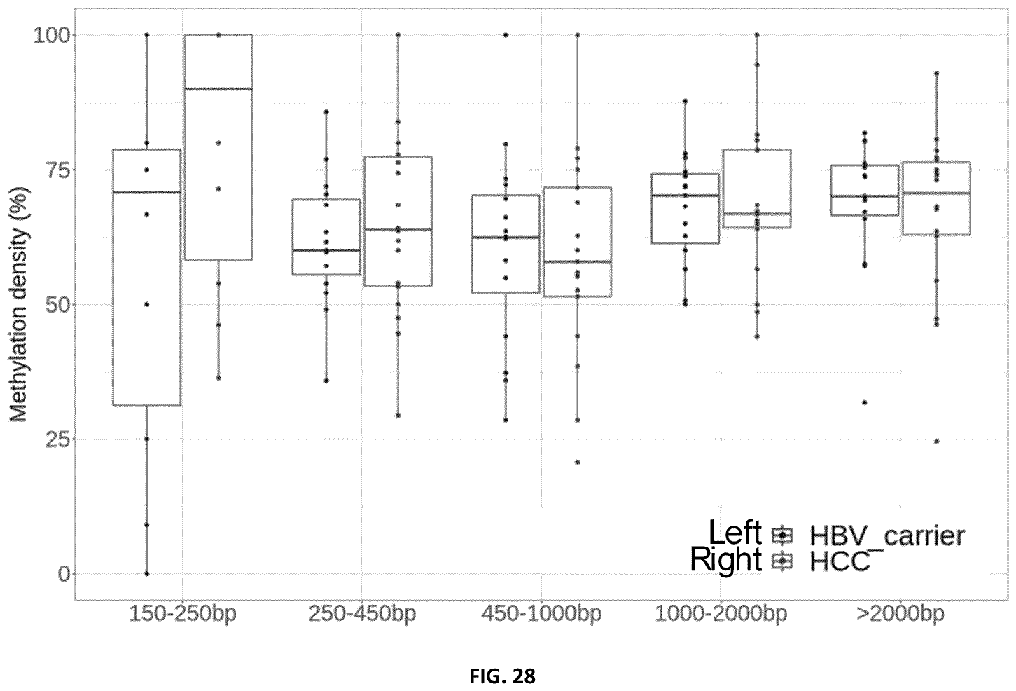

FIG. 28 presents a graph illustrating methylation density values calculated for eccDNA remnants containing at least one CpG site. The vertical y-axis of the graph represents the methylation density values for eccDNA remnants having a length longer than 1 kb, and the horizontal x-axis lists several different eccDNA remnant size ranges. For each of these different size ranges, the eccDNA remnant methylation density for HBV carrier samples (left) and HCC patient samples (right) are plotted. As shown in FIG. 28, the methylation density of the eccDNA remnants from both HBV carriers and HCC patients generally exhibited a decrease within the 150 bp to 1000 bp range. The median methylation density of eccDNA remnants in size ranges of 150-250 bp, 250-450 bp and 450-1000 bp from HBV carriers were 70.8%, 60.0%, and 62.4%, respectively, whereas those from HCC patients were 90.0%, 63.8% and 57.9%. Conversely, the methylation density of eccDNA remnants increased with the length exceeding 1000 bp. For eccDNA remnants in size ranges of 1000-2000 bp and >2000 bp from HBV carriers, the median methylation densities were 70.2% and 70.1%, respectively, compared to 66.8% and 70.7% for those from HCC patients. This observed pattern suggests variations in the original fragment lengths of the eccDNA remnants.

The data of FIG. 28 further shows that for smaller eccDNA fragments, for example those in the size ranges of 150-250 bp and 250-450 bp, the methylation density values of eccDNA fragments from HCC patient samples are higher than those of eccDNA fragments from HBV carrier samples. Additionally, for larger eccDNA fragments, for example those in the size range of 1000-2000 bp, the methylation density values of eccDNA fragments from HCC patient samples are higher than those of eccDNA fragments from HBV carrier samples. These findings suggest the presence of one or more cutoff or threshold size values related to eccDNA methylation differences associated with a cancer state. For example, the results indicate the presence of an upper cutoff value below which the eccDNA remnants from cancer samples have a higher methylation density than that of eccDNA remnants from non-cancer samples. The results also indicate the presence of a lower cutoff value above which the eccDNA remnants from cancer samples have a lower methylation density than that of eccDNA remnants from non-cancer samples.

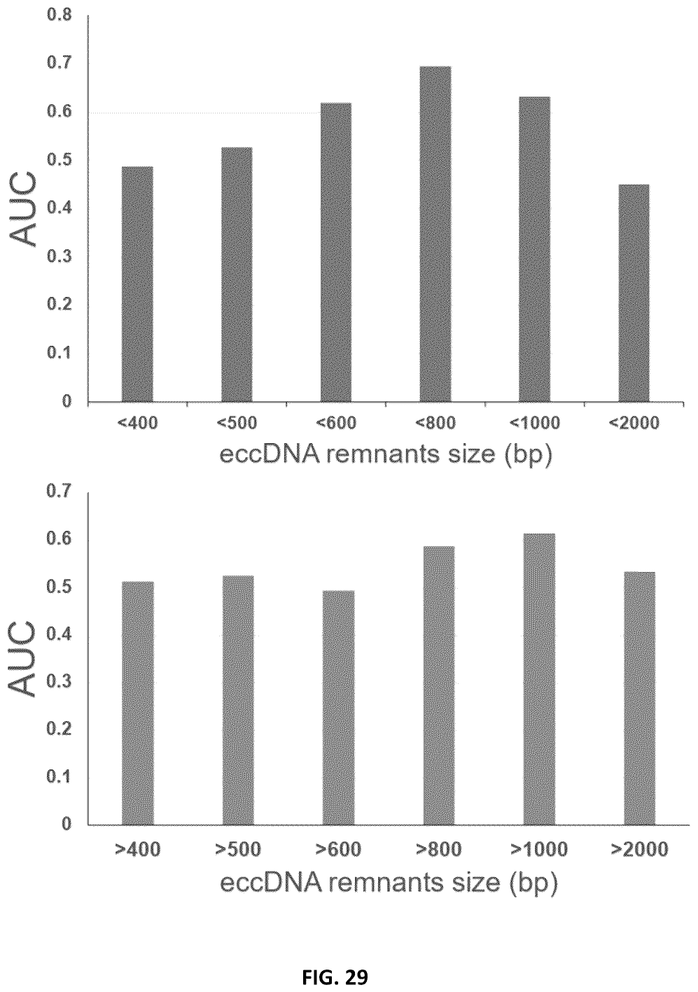

FIG. 29 presents a pair of graphs showing area under the curve (AUC) values related to using methylation density values to differentiate eccDNA remnants of different sizes from HCC patient samples and HBV carrier samples. The vertical y-axes of the graphs represents the AUC values for distinguishing the two sample types, and the horizontal x-axes lists several different eccDNA remnant size ranges based on upper cutoff values (upper graph) or lower cutoff values (lower graph). The data of the upper graph show that HCC can be detected using methylation density values for eccDNA remnants having sizes that are less than 600 bp, less than 800 bp, or less than 1000 bp. The data further suggest that, in terms of upper size limits, the most accurate cancer detection based on eccDNA remnant methylation densities is achieved with eccDNA remnants having an upper size limit that is between about 600 bp and about 1000 bp (e.g., an upper size limit of about 800 bp). The data of the lower graph show that HCC can be detected using methylation density values for eccDNA remnants having sizes that are greater than 800 bp, greater than 1000 bp, or greater than 2000 bp. The data further suggest that, in terms of lower size limits, the most accurate cancer detection based on eccDNA remnant methylation densities is achieved with eccDNA remnants having a lower size limit that is between about 800 bp and about 2000 bp (e.g., a lower size limit of about 1000 bp.)

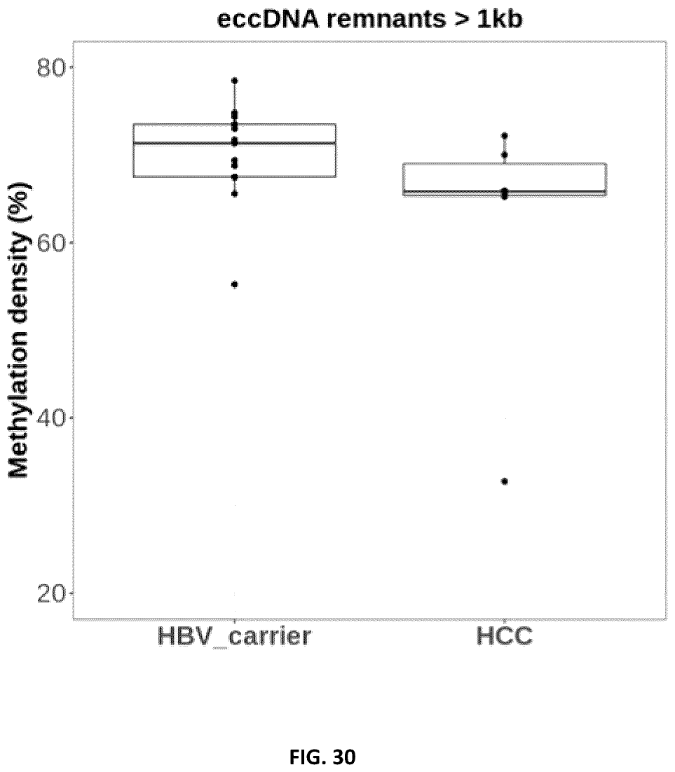

FIG. 30 shows a graph applying an example of a lower eccDNA remnant size limit to detect cancer using methylation density values. The vertical y-axis of the graph represents the methylation density percentage for eccDNA remnants having a length longer than 1 kb, and the data points indicate percentage values for different HCC patient samples (left) and HBV carrier samples (right). More specifically, this example involved analysis of 5 HCC samples with tumor DNA fraction of at least 20%, and 14 samples without HCC. As shown in FIG. 30, the median methylation densities for eccDNA remnants larger than 1 kb differed between non-HCC (71.3%) and HCC (65.8%) samples. This difference demonstrates that large eccDNA remnants exhibit hypomethylation in HCC cases and can therefore provide a distinguishing feature for cancer detection, e.g., the identification and differentiation of HCC samples from non-HCC samples.

Together, these results thus demonstrate the ability of the provided methods to detect and classify eccDNA remnants in biological samples by using linear sequencing data. The results further demonstrate how an analysis of collective properties of these detected eccDNA remnants, such as their counts, size distributions, genomic distributions, nucleotide motif patterns, and/or methylation profiles can be used to classify a pathology, and determine a cancer level in a subject.

### E. Method

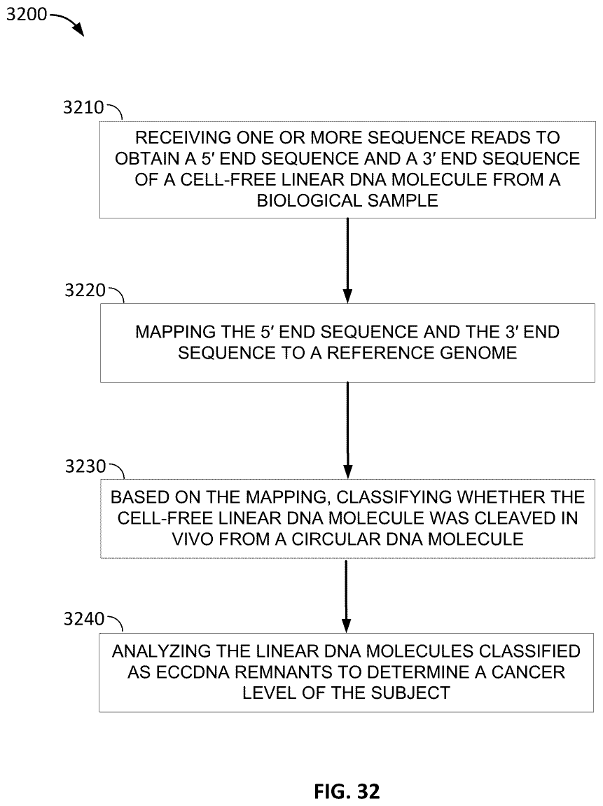

FIG. 32 presents a flowchart of a method 3200 for analyzing a biological sample from a subject to determine a cancer level of a subject based on an analysis of eccDNA remnants according to embodiments of the present disclosure. Various examples of operations of method 3200 are described in Sections II, III, and 0. Method 3200 can be performed partially or entirely using a computer system.

At block 3210, one or more sequence reads are obtained, where the one or more sequence reads include a 5′ end sequence of a linear DNA molecule in a biological sample, and a 3′ end sequence of the linear DNA molecule. Block 3210 can be performed in a similar manner to block 410 of method 400, presented in FIG. 4, and block 510 of method 500, presented in FIG. 5. As with blocks 410 and 510, in some examples, obtaining the sequence reads includes receiving the biological sample. The biological sample may be obtained from a subject being screened for cancer. The biological sample can include cell-free DNA. For example, the biological sample can be one that is purified, e.g., to separate out a predominantly cell-free portion, such as plasma. Other pre-processing steps may be performed with the biological sample as well. In some examples, obtaining the sequencing reads includes sequencing the linear DNA molecules present in the sample. The sequencing can be a random sequencing of all the linear DNA molecules in the biological sample, rather than a targeted sequencing of particular molecules, and can be performed using any of the sequencing techniques described in Section IIA. In some instances, the biological sample is a cell-free biological sample and/or the linear DNA molecules are cell-free linear DNA molecules. For example, the biological sample can include or consist of plasma. The linear DNA molecules of the biological sample are naturally found in the biological sample in a linear form, and the provided method does not include operations, such as enzymatic cleavage or mechanical shearing, intended to linearize circular DNA molecules of the biological sample. In some instances, one obtained sequence read includes both the 5′ end sequence and the 3′ end sequence of a linear DNA molecule. In other instances, one obtained sequence read includes the 5′ end sequence of a linear DNA molecule, and another obtained sequence read includes the 3′ end sequence of the linear DNA molecule. The obtained sequence reads can each independently have a length that is at least 25 bp, at least 45 bp, at least 75 bp, at least 150 bp, at least 250 bp, at least 500 bp, at least 1 kb, at least 3 kb, at least 10 kb, at least 30 kb, or at least 100 kb

At block 3220, the 5′ end sequence of the linear DNA molecule and the 3′ end sequence of the linear DNA molecule obtained in block 3210 are mapped to a reference genome. Block 3220 can be performed in a similar manner to block 420 of method 400, and block 520 of method 500. As with blocks 420 and 520, the mapping can be performed as described in Section IIA, and can involve independently determining an optimal alignment for each of the 5′ end sequence and the 3′ end sequence to the reference genome. In some instances, the alignments are each independently required to satisfy a mapping quality condition, for example, having a mapping quality that is at least 20, at least 25, at least 30, at least 35, at least 40, at least 45, or at least 50. In some cases, the mapping of the end sequences includes determining a genomic coordinate for each of the end sequences. Additionally or alternatively, the mapping of the end sequences can include determining a genomic orientation for each of the end sequences. In some examples, the provided method also includes additionally mapping one or more sequences of the linear DNA molecule other than the 5′ end sequence and the 3′ end sequence, and determining the genomic coordinates and/or genomic orientations of these additionally mapped sequences. In some instances, the mapping further includes identifying if a sequence read includes a junction locus, and optionally determining the genomic coordinate of this junction.

At block 3230, the linear DNA molecule is classified, based on the mapping in block 3220, according to whether or not the linear DNA molecule was cleaved in vivo from a circular DNA molecule, i.e., whether or not the linear DNA molecule is an eccDNA remnant. Block 3230 can be performed in a similar manner to block 430 of method 400, and block 530 of method 500. As with blocks 430 and 530, the classifying of the linear DNA molecule can be performed as described in Section IIB. For instance, the classifying can include comparing the genomic coordinate of the 5′ end sequence of the linear DNA molecule to the genomic coordinate of the 3′ end sequence of the linear DNA molecule. Additionally or alternatively, the classifying can include comparing the genomic orientation of the 5′ end sequence to the genomic orientation of the 3′ end sequence. As one example, the linear DNA molecule can be classified as an eccDNA remnant if (1) the genomic coordinate of the 5′ end sequence is larger than the genomic coordinate of the 3′ end sequence, and (2) the genomic orientation of the 5′ end sequence is identical to the genomic orientation of the 3′ end sequence. In some cases, the classifying can include comparisons of genomic coordinates and/or genomic orientations of mapped sequences other than or in addition to the 5′ end sequence and the 3′ end sequence.

At block 3240, the members of a set of all linear DNA molecules classified as being an eccDNA remnant in block 3230 are analyzed to determine a cancer level of the subject from whom the biological sample was obtained. In some instances, all members of the set of classified eccDNA remnants are analyzed. In other instances, a portion of the set of classified eccDNA remnants are analyzed. The analyses of the eccDNA remnants can be used in some examples to determine a collective value of the set of eccDNA remnants. One example of such a collective property is a count, e.g., an absolute count or a relative or normalized count, of eccDNA remnants in the set. Another example of such a collective property is a size distribution of eccDNA remnants in the set. Another example of such a collective property is a genomic distribution of eccDNA remnants in the set. Another example of such a collective property is a frequency of one or more nucleotide motif patters occurring for the eccDNA remnants in the set. Another example of such a collective property is a methylation status for the eccDNA remnants in the set that have a size within a specified size range.

The collective value can be used to determine the cancer level of the subject. In some cases, a count of the set of eccDNA remnants is used to determine the cancer level of the subject, for example by comparing the count with a reference value and determining if the count is greater than, less than, or equal to the reference value. In other cases, a size distribution of the set of eccDNA remnants is used to determine the cancer level of the subject, for example by determining a percentage of the set of eccDNA remnants that exceed a size threshold, e.g., a predetermined size threshold, and optionally then determining if this percentage is greater than, less than, or equal to a reference percentage value. In still other cases, determining the cancer level of the subject includes using a normalized genomic coverage of the eccDNA remnants, for example where the normalized genomic coverage is calculated by counting a subset of eccDNA fragments mapping to regions within a class of genomic elements, and dividing this count by the percentage of the genome withing the regions. In some cases, determining the cancer level of the subject includes using a frequency of one or more nucleotide motif patterns occurring for the set of eccDNA remnants, for example where at least one of the nucleotide motif patterns includes a plurality of nucleotide motifs, e.g., trinucleotide motifs, dinucleotide motifs, or tetranucleotide motifs. In other cases, determining the cancer level of the subject includes using a methylation status of eccDNA remnants having a size within a specified size range, for example, a size less than a maximum size (e.g., 1000 bp) or a size greater than a minimum size (e.g., 800 bp).

### VI. Methods for Determining a Fractional DNA Concentration

In some aspects, the present disclosure provides various methods for determining a fractional concentration of DNA of interest, e.g., clinically relevant DNA, in a biological sample from a subject, where the fractional concentration is determined by analyzing eccDNA remnants in the biological sample as described in Section IV.A. The DNA of interest could be, for example, DNA that is specifically from a tumor tissue, a transplant tissue, or fetal tissue. Because previous approaches for determining fractional DNA concentrations did not consider or analyze eccDNA remnants present in a biological sample, the methods disclosed herein advantageously provide a new source of information that can be useful in providing more accurate assessments of fractional DNA concentrations through noninvasive sampling and assays.

As an example, nanopore sequencing was performed using cell-free DNA (cfDNA) extracted from the plasma of nine HCC patients. An analysis following alignment of the resulting sequence reads to a reference genome found a median of 52,774,950 mapped fragments for the samples, with a range from 22,475,592 to 147,920,695 mapped fragments. Subsequently, ichorCNA (Adalsteinsson et al. Nat Commun. 2017; 8:1324) was used to estimate the tumor fraction in the nine HCC patients, finding tumor fractions ranging from 7.95% to 47.5%. Using the methods described in Sections II.C and III.F, eccDNA remnants were then classified in each sample from the HCC subjects based on the mapping of the sequence reads. The sizes and size distributions of the eccDNA remnants in each of the biological samples were also determined as described in Section III.B, and the determined eccDNA remnant size distributions for the biological samples were used to calculate the percentage of eccDNA remnants larger than 1 kb in each sample.

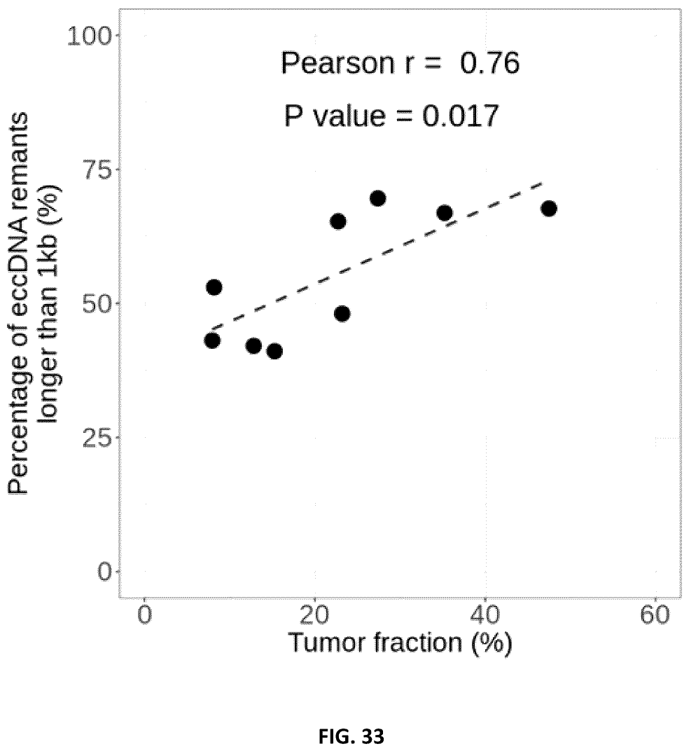

FIG. 33 presents a graph comparing the percentage of these large (i.e., >1 kb) eccDNA fragments in each sample with the tumor fraction for the sample. The vertical y-axis of the graph represents the percentage of eccDNA remnants having a length longer than 1 kb, and the horizontal x-axis represents the tumor fraction, with data points plotted for each of the nine HCC samples. Notably, a positive correlation was observed between the percentage of eccDNA remnants longer than 1 kb and the tumor fraction (Pearson's r=0.76; p-value 0.017). These findings demonstrate that eccDNA remnants in certain sizes (e.g. >1 kb) can reflect the fractional concentration of clinically relevant DNA.

### A. Use of Calibration Data Points

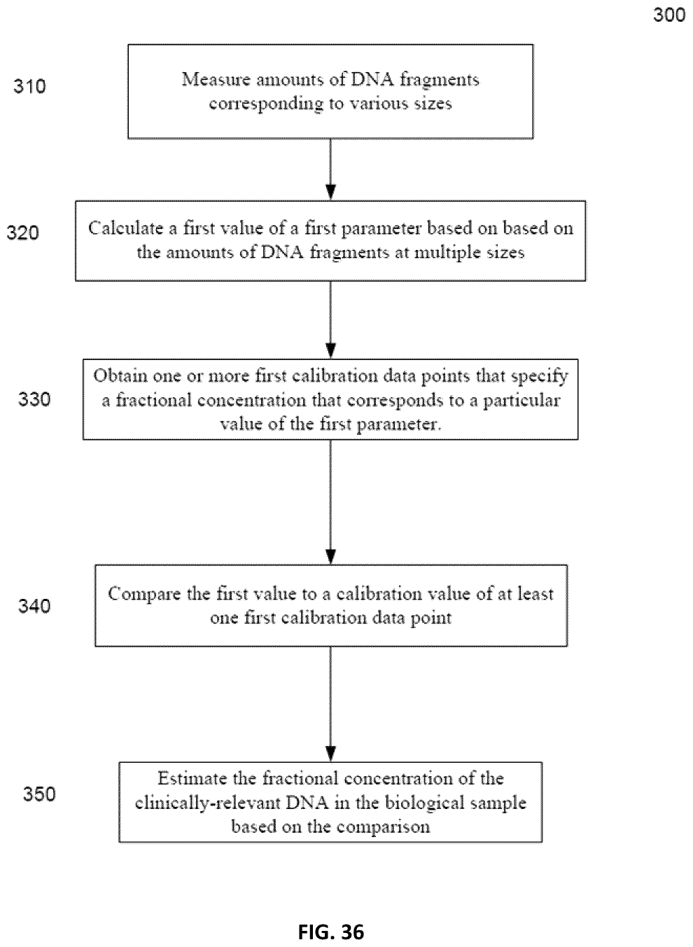

FIG. 36 is a flowchart of a method 300 illustrating a method of estimating a fractional concentration of clinically-relevant DNA in a biological sample according to embodiments of the present invention. The biological sample includes the clinically-relevant DNA and other DNA. The biological sample may be obtained from a patient, e.g., a female subject pregnant with a fetus. In another embodiment, the patient may have or be suspected of having a tumor. In one implementation, the biological sample may be received at a machine, e.g., a sequencing machine, which outputs measurement data (e.g., sequence reads) that can be used to determine sizes of the DNA fragments. Method 300 may be performed wholly or partially with a computer system, as can other methods described herein.

At block 310, amounts of eccDNA remnants corresponding to various characteristic values (e.g., sizes, number of remnants, methylation density, or motif number) are measured. For each size of a plurality of sizes, an amount of a plurality of DNA fragments from the biological sample corresponding to the size can be measured. For instance, the number of DNA fragments having a length of greater than 100 bp, greater than 300 bp, greater than 1 kb, greater than 3 kb, greater than 10 kb, greater than 30 kb, and/or greater than 100 kb may be measured. The amounts may be saved as a histogram. In one embodiment, a size of each of the plurality of nucleic acids from the biological sample is measured, which may be done on an individual basis (e.g., by single molecule sequencing) or on a group basis (e.g., via electrophoresis). The sizes may correspond to a range. Thus, an amount can be for DNA fragments that have a size within a particular range.

The plurality of eccDNA remnants may be chosen at random or preferentially selected from one or more predetermined regions of a genome. For example, targeted enrichment may be performed, as described above. In another embodiment, eccDNA remnants may be randomly sequenced (e.g., using universal sequencing), and the resulting sequence reads can be aligned to a genome corresponding to the subject (e.g., a reference human genome). Then, eccDNA remnants whose sequence reads align to the one or more predetermined regions may be used to determine the size.

In various embodiments, the size can be mass, length, or other suitable size measures. The measurement can be performed in various ways, as described herein. For example, paired-end sequencing and alignment of eccDNA remnants may be performed, or electrophoresis may be used. A statistically significant number of eccDNA remnants can be measured to provide an accurate size profile of the biological sample. Examples of a statistically significant number of eccDNA remnants include greater than 100,000; 1,000,000; 2,000,000, or other suitable values, which may depend on the precision required.

In one embodiment, the data obtained from a physical measurement, such as paired-end sequencing or electrophoresis, can be received at a computer and analyzed to accomplish the measurement of the sizes of the eccDNA remnants. For instance, the sequence reads from the paired-end sequencing can be analyzed (e.g., by alignment) to determine the sizes. As another example, the electropherogram resulting from electrophoresis can be analyzed to determines the sizes. In one implementation, the analyzing of the eccDNA remnants does include the actual process of sequencing or subjecting eccDNA remnants to electrophoresis, while other implementations can just perform an analysis of the resulting data.

At block 320, a first value of a first parameter is calculated based on the amounts of eccDNA remnants at multiple sizes. In one aspect, the first parameter provides a statistical measure of a size profile (e.g., a histogram) of DNA fragments in the biological sample. The parameter may be referred to as a size parameter if it is determined from the sizes of the plurality of eccDNA remnants.

The first parameter can be of various forms. Such a parameter is a number of eccDNA remnants having a particular characteristic value divided by the total number of fragments, which may be obtained from a histogram (any data structure providing absolute or relative counts of fragments having particular characteristic values). As another example, a parameter could be a number of fragments having a particular characteristic value or within a particular range divided by a number of fragments having another characteristic value or range. The division can act as a normalization to account for a different number of eccDNA remnants being analyzed for different samples. A normalization can be accomplished by analyzing a same number of eccDNA remnants for each sample, which effectively provides a same result as dividing by a total number of fragments analyzed. Other examples of parameters are described herein.

At block 330, one or more first calibration data points are obtained. Each first calibration data point can specify a fractional concentration of clinically-relevant DNA corresponding to a particular value (a calibration value) of the first parameter. The fractional concentration can be specified as a particular concentration or a range of concentrations. A calibration value may correspond to a value of the first parameter (e.g., a particular size parameter) as determined from a plurality of calibration samples. The calibration data points can be determined from calibration samples with known fractional concentrations, which may be measured via various techniques described herein. At least some of the calibration samples would have a different fractional concentration, but some calibration samples may have a same fractional concentration

In various embodiments, one or more calibration points may be defined as one discrete point, a set of discrete points, as a function, as one discrete point and a function, or any other combination of discrete or continuous sets of values. As an example, a calibration data point could be determined from one calibration value of a size parameter (e.g., number of fragments in a particular size or size range) for a sample with a particular fractional concentration. A plurality of histograms can be used, with a different histogram for each calibration sample, where some of the calibration samples may have the same fractional concentration.

In one embodiment, measured values of a same parameter from multiple samples at the same fractional concentration could be combined to determine a calibration data point for a particular fractional concentration. For example, an average of the values of the parameter may be obtained from the data of samples at the same fractional concentration to determine a particular calibration data point (or provide a range that corresponds to the calibration data point). In another embodiment, multiple data points with the same calibration value can be used to determine an average fractional concentration.

In one implementation, the characteristic values of eccDNA remnants are measured for many calibration samples. A calibration value of the same parameter is determined for each calibration sample, where the parameter may be plotted against the known fractional concentration of the sample. A function may then be fit to the data points of the plot, where the functional fit defines the calibration data points to be used in determining the fractional concentration for a new sample.

At block 340, the first value is compared to a calibration value of at least one calibration data point. The comparison can be performed in a variety of ways. For example, the comparison can be whether the first value is higher or lower than the calibration value. The comparison can involve comparing to a calibration curve (composed of the calibration data points), and thus the comparison can identify the point on the curve having the first value of the first parameter. For example, a calculated value X of the first parameter (as determined from the measured characteristic values of DNA in the new sample) can be used as input into a function F(X), where F is the calibration function (curve). The output of F(X) is the fractional concentration. An error range can be provided, which may be different for each X value, thereby providing a range of values as an output of F(X).

In step 350, the fractional concentration of the clinically-relevant DNA in the biological sample is estimated based on the comparison. In one embodiment, one can determine if the first value of the first parameter is above or below a threshold calibration value, and thereby determine if the estimated fractional concentration of the instant sample is above or below the fractional concentration corresponding to the threshold calibration value. For example, if the calculated first value X₁ for the biological is above a calibration value X_{C} then the fractional concentration FC₁ of the biological sample can be determined as being above the fractional concentration FC_{C} corresponding to X_{C}. This comparison can be used to determine if a sufficient fractional concentration exists in the biological sample to perform other tests, e.g., testing for a fetal aneuploidy. This relationship of above and below can depend on how the parameter is defined. In such an embodiment, only one calibration data point may be needed.

In another embodiment, the comparison is accomplished by inputting the first value into a calibration function. The calibration function can effectively compare the first value to calibration values by identifying the point on a curve corresponding to the first value. The estimated fractional concentration is then provided as the output value of the calibration function.

In one embodiment, the value of more than one parameter can be determined for the biological sample. For example, a second value can be determined for a second parameter, which corresponds to a different statistical measure of the size profile of eccDNA remnants in the biological sample. The second value can be determined using the same characteristic value measurements of the DNA fragments, or different characteristic value measurements. Each parameter can correspond to a different calibration curve. In one implementation, the different values can be compared independently to different calibration curves to obtain a plurality of estimated fractional concentrations, which may then be averaged or used to provide a range as an output.

In another implementation, a multidimensional calibration curve can be used, where the different values of the parameters can effectively be input to a single calibration function that outputs the fractional concentration. The single calibration function can result from a functional fit of all of the data points obtained from the calibration samples. Thus, in one embodiment, the first calibration data points and the second calibration data points can be points on a multidimensional curve, where the comparison includes identifying the multidimensional point having coordinates corresponding to the first value and the one or more second values

### B. Determining Calibration Data Points

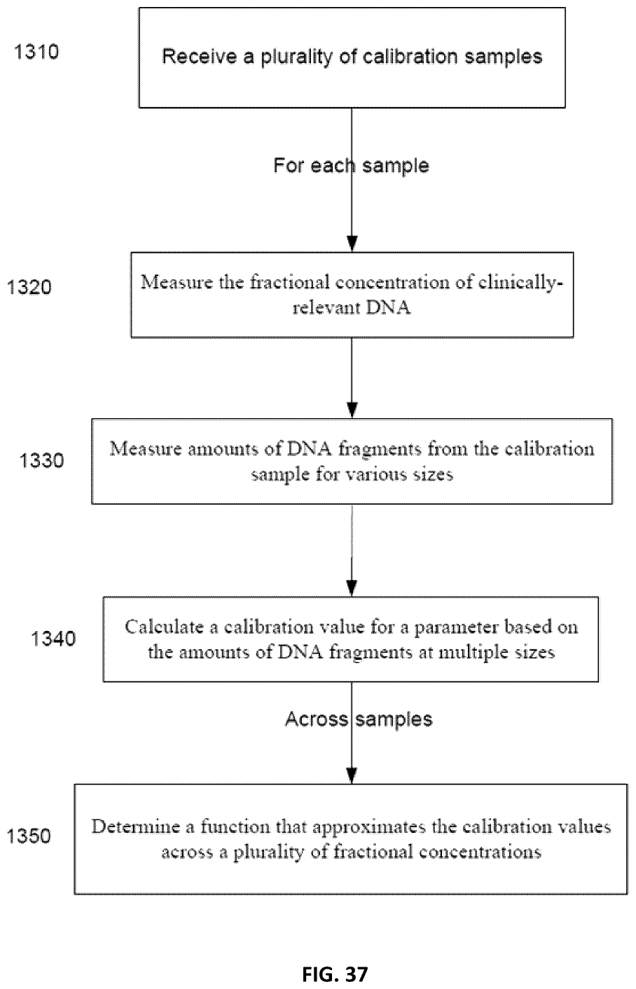

FIG. 37 is a flowchart of a method 1300 for determining calibration data points from measurements made from calibration samples according to embodiments of the present invention. The calibration samples include the clinically-relevant DNA and other DNA.

At block 1310, a plurality of calibration samples are received. The calibration samples may be obtained as described herein. Each sample can be analyzed separately via separate experiments or via some identification means (e.g., tagging an eccDNA remnant with a bar code) to identify which sample a molecule was from. For example, a calibration sample may be received at a machine, e.g., a sequencing machine, which outputs measurement data (e.g., sequence reads) that can be used to determine characteristic values of the eccDNA remnants, or is received at an electrophoresis machine.

At block 1320, the fractional concentration of clinically-relevant DNA is measured in each of the plurality of calibration samples. In various embodiments measuring a fetal DNA concentration, a paternally-inherited sequence, or a fetal-specific epigenetic markers may be used. For example, a paternally-inherited allele would be absent from a genome of the pregnant female and can be detected in maternal plasma at a percentage that is proportional to the fractional fetal DNA concentration. Fetal-specific epigenetic markers can include DNA sequences that exhibit fetal or placental-specific DNA methylation patterns in maternal plasma.

At block 1330, amounts of eccDNA remnants from each calibration sample are measured for various characteristic values, e.g., sizes, number of remnants, methylation density, or motif number. The characteristic values may be measured as described herein. The characteristic values may be counted, plotted, used to create a histogram, or other sorting procedure to obtain data regarding a characteristic value profile of the calibration sample.

At block 1340, a calibration value is calculated for a parameter based on the amounts of DNA fragments at multiple characteristic values, e.g., sizes. A calibration value can be calculated for each calibration sample. In one embodiment, the same parameter is used for each calibration value. However, embodiments may use multiple parameters as described herein. For example, the cumulative fraction of eccDNA remnants less than 1000 bases may be used as the parameter, and samples with different fractional concentration would likely have different calibration values. A calibration data point may be determined for each sample, where the calibration data point includes the calibration value and the measured fractional concentration for the sample. These calibration data points can be used in method 300, or can be used to determine the final calibration data points (e.g., as defined via a functional fit).

At block 1350, a function that approximates the calibration values across a plurality of fractional concentrations is determined. For example, a linear function could be fit to the calibration values as a function of fractional concentration. The linear function can define the calibration data points to be used in method 300.

In some embodiments, calibration values for multiple parameters can be calculated for each sample. The calibration values for a sample can define a multidimensional coordinate (where each dimension is for each parameter) that along with the fractional concentration can provide a data point. Thus, in one implementation, a multidimensional function can be fit to all of the multidimensional data points. Accordingly, a multidimensional calibration curve can be used, where the different values of the parameters can effectively be input to a single calibration function that outputs the fractional concentration. And the single calibration function can result from a functional fit of all of the data points obtained from the calibration samples.

### VII. Treatments

### A. Further Screening Modalities

Based on any classification, e.g., regarding a pathology or fractional concentration of clinically-relevant DNA, the subject can be referred for additional screening modalities, e.g. using chest X ray, ultrasound, computed tomography, magnetic resonance imaging, or positron emission tomography. Such screening may be performed for cancer.

### B. Treatment Selection

Embodiments of the present disclosure can accurately predict disease relapse, thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell transplant, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### VIII. Systems

In another aspect, the present disclosure provides various systems, e.g., measurement systems and/or computer systems, for performing the methods described herein, or individual or combined operations of those methods.

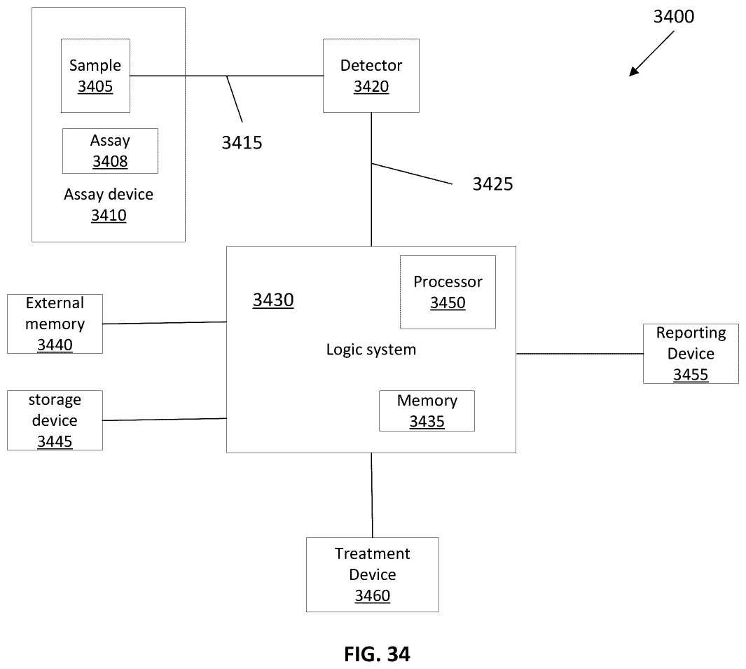

FIG. 34 illustrates a measurement system 3400 according to an embodiment of the present disclosure. The system as shown includes a sample 3405, such as cell-free DNA molecules within an assay device 3410, where an assay 3408 can be performed on sample 3405. For example, sample 3405 can be contacted with reagents of assay 3408 to provide a signal of a physical characteristic 3415 (e.g., sequences of linear DNA molecules). An example of an assay device can be a flow cell that includes probes and/or primers of an assay or a tube through which a droplet moves (with the droplet including the assay). Physical characteristic 3415 (e.g., a fluorescence intensity, a voltage, or a current), from the sample is detected by detector 3420. Detector 3420 can take a measurement at intervals (e.g., periodic intervals) to obtain data points that make up a data signal. In one embodiment, an analog-to-digital converter converts an analog signal from the detector into digital form at a plurality of times. Assay device 3410 and detector 3420 can form an assay system, e.g., sequencing device that sequences linear DNA molecules from biological samples according to embodiments described herein. A data signal 3425 is sent from detector 3420 to logic system 3430. As an example, data signal 3425 can be used to determine sequence information. Data signal 3425 can include various measurements made at a same time, e.g., different colors of fluorescent dyes or different electrical signals for a different molecule of sample 3405, and thus data signal 3425 can correspond to multiple signals. Data signal 3425 may be stored in a local memory 3435, an external memory 3440, or a storage device 3445.

Logic system 3430 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 3430 and the other components may be part of a stand-alone or network connected computer system, or they may be directly attached to or incorporated in a device (e.g., a sequencing device) that includes detector 3420 and/or assay device 3410. Logic system 3430 may also include software that executes in a processor 3450. Logic system 3430 may include a computer readable medium storing instructions for controlling measurement system 3400 to perform any of the methods described herein. For example, logic system 3430 can provide commands to a system that includes assay device 3410 such that sequencing operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 3400 may also include a reporting device 3455, which can present results of any of the methods describe herein, e.g., as determined using the measurement system. Reporting device 3455 can be in communication with a reporting module within logic system 3430 that can aggregate, format, and send a report to reporting device 3455. Reporting device 3455 can present information indicating, for example, characteristics of molecules classified as eccDNA remnants in sample 3405, where the characteristics can advantageously provide information related to the biological sample or to the subject from which the sample was derived. The reporting module can present information from any one or more of the detecting and/or determining steps in methods 400, 500, and/or 1100, as described in Sections II.C, III.C, and V.B, respectively. The information can be presented by reporting device 3455 in any format that can be recognized and interpreted by a user of the measurement system 3400. For example, the information can be presented by reporting device 3455 in a displayed, printed, or transmitted format, or any combination thereof.

Measurement system 3400 may also include a treatment device 3460, which can provide a treatment to the subject. Treatment device 3460 can determine a treatment and/or be used to perform a treatment. Examples of such treatment can include surgery, radiation therapy, chemotherapy, immunotherapy, targeted therapy, hormone therapy, and stem cell transplant. Logic system 3430 may be connected to treatment device 3460, e.g., to provide results of a method described herein. The treatment device may receive inputs from other devices, such as an imaging device and user inputs (e.g., to control the treatment, such as controls over a robotic system).

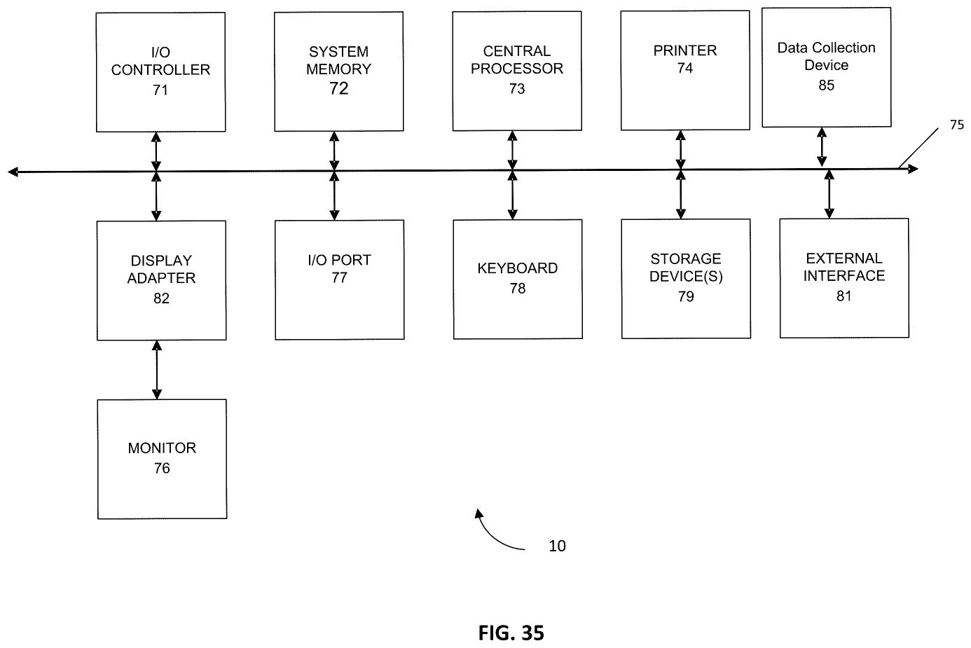

Any of the computer systems mentioned herein may utilize any suitable number of subsystems. Examples of such subsystems are shown in FIG. 35 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 35 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FIREWIRE®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communications between devices, including at least 100, 200, 500, 1,000, 10,000, 50,000, 100,000, 500,00, or one million communications. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor may be performed in real-time. The term “real-time” may refer to computing operations or processes that are completed within a certain time constraint. The time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a,” “an,” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location or order unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted as being prior art. Where a conflict exists between the instant application and a reference provided herein, the instant application shall dominate.

What is claimed is:

1. A method for analyzing a biological sample from a subject, the biological sample comprising a plurality of cell-free linear DNA molecules that each independently comprise a 5′ end sequence and a 3′ end sequence, the method comprising:
for each of the plurality of cell-free linear DNA molecules:
receiving one or more sequence reads comprising at least the 5′ end sequence and the 3′ end sequence to obtain a 5′ end sequence read and a 3′ end sequence read;
mapping the 5′ end sequence and the 3′ end sequence to a reference genome; and
based on the mapping, classifying whether the cell-free linear DNA molecule was cleaved in vivo from a circular DNA molecule, thereby identifying a set of eccDNA remnants; and
analyzing the set of eccDNA remnants to determine a property of the biological sample or subject.

2. The method of claim 1, wherein the mapping of the 5′ end sequence and the 3′ end sequence to the reference genome comprises:
determining a genomic coordinate of the 5′ end sequence and a genomic orientation of the 5′ end sequence; and
determining a genomic coordinate of the 3′ end sequence and a genomic orientation of the 3′ end sequence.

3. The method of claim 2, wherein the classifying comprises:
comparing the genomic coordinate of the 5′ end sequence to the genomic coordinate of the 3′ end sequence; and
comparing the genomic orientation of the 5′ end sequence to the genomic orientation of the 3′ end sequence.

4. The method of claim 3, wherein the classifying further comprises:
classifying the cell-free linear DNA molecule as a member of the set of eccDNA remnants if both
(1) the genomic coordinate of the 5′ end sequence is larger than the genomic coordinate of the 3′ end sequence, and
(2) the genomic orientation of the 5′ end sequence is identical to the genomic orientation of the 3′ end sequence.

5. The method of claim 1, wherein, for at least a portion of the eccDNA remnants, the 5′ end sequence read and the 3′ end sequence read do not comprise a junction at which nucleotides at two separated genomic locations are immediately adjacent to one another.

6. The method of claim 1, wherein, for each of the plurality of cell-free linear DNA molecules independently, one sequence read comprises the 5′ end sequence and the 3′ end sequence.

7. The method of claim 1, wherein the mapping of the 5′ end sequence and the 3′ end sequence to the reference genome are each independently based on satisfying a mapping quality condition.

8. The method of claim 1, wherein the analyzing of the set of eccDNA remnants comprises:
determining a count of the set of eccDNA remnants; and
using the count to determine the property of the biological sample or subject.

9. The method of claim 8, wherein the using of the count comprises comparing the count to a reference value.

10. The method of claim 8, wherein the count of the set of eccDNA remnants is normalized with respect to a count of the cell-free linear DNA molecules in the plurality of cell-free linear DNA molecules.

11. The method of claim 1, wherein the analyzing of the set of eccDNA remnants comprises:
determining a size distribution of original eccDNA molecules corresponding to the set of eccDNA remnants; and
using the size distribution to determine the property of the biological sample or subject.

12. The method of claim 11, wherein:
for at least a portion of the eccDNA remnants, at least one of the 5′ end sequence read and the 3′ end sequence read comprises a junction at which nucleotides at two separated genomic locations are immediately adjacent to one another, and
the determining of the size distribution comprises, for each of eccDNA remnants for which the 5′ end sequence read or the 3′ end sequence read comprises the junction, calculating a distance between the two separated genomic locations.

13. The method of claim 11, wherein a statistical value of the size distribution is used to determine the property.

14. The method of claim 13, wherein the statistical value is a percentage of the set of eccDNA remnants that exceed a size threshold.

15. The method of claim 1, wherein the analyzing of the set of eccDNA remnants comprises:
for each eccDNA remnant of the set of eccDNA remnants, determining, based on the mapping, whether the eccDNA remnant is a member of a subset of the eccDNA remnants, wherein each member of the subset of the eccDNA remnants maps to one or more regions within a class of genomic elements of the reference genome;
determining a count of the subset of the eccDNA remnants;
normalizing the count with respect to a total size of the one or more regions in the reference genome; and
using the normalized count to determine the property of the biological sample or subject.

16. The method of claim 15, wherein normalizing the count comprises dividing the count by the percentage of the reference genome within the one or more regions.

17. The method of claim 15, wherein the class of genomic elements comprises 5′ untranslated regions, 3′ untranslated regions, exons, introns, regions 2 kb upstream of genes (Gene2kbU), regions 2 kb downstream of genes (Gene2kbD), CpG islands, regions 2 kb upstream of CpG islands (CGI2kbU), regions 2 kb downstream of CpG islands (CGI2kbD), Alu repeat regions, or a combination thereof.

18. The method of claim 1, wherein the analyzing of the set of eccDNA remnants comprises:
determining a frequency of one or more nucleotide motif patterns occurring for the set of eccDNA remnants, each nucleotide motif pattern comprising one or more nucleotide motifs, each nucleotide motif located in a respective segment of the reference genome or of the eccDNA remnant, each segment independently within a specified distance from either a respective 5′ end of an eccDNA remnant of the set of eccDNA remnants or a respective 3′ end sequence of the eccDNA remnant; and
using the frequency to determine the property of the biological sample or subject.

19. The method of claim 18, wherein the specified distance is 50 bp.

20. The method of claim 18, wherein at least one of the one or more nucleotide motif patterns comprises a plurality of nucleotide motifs.

21. The method of claim 18, wherein each nucleotide motif is independently a trinucleotide motif, a dinucleotide motif, or a tetranucleotide motif.

22. The method of claim 1, wherein the analyzing of the set of eccDNA remnants comprises:
identifying a subset of the set of eccDNA remnants, each eccDNA remnant of the subset independently having a size within a specified size range;
for each eccDNA remnant of the subset, determining a methylation status at one or more sites of the eccDNA remnant;
based on the determined methylation statuses, determining a methylation density for the subset; and
using the methylation density to determine the property of the biological sample or subject.

23. The method of claim 22, wherein each eccDNA remnant of the subset independently has a size less than a maximum size, wherein the maximum size is optionally 1000 bp.

24. The method of claim 22, wherein each eccDNA remnant of the subset independently has a size greater than a minimum size, wherein the minimum size is optionally 800 bp.

25. The method of claim 1, wherein the property of the subject comprises a level of a cancer.

26. The method of claim 1, wherein the property of the subject comprises a fractional concentration of DNA of interest.

27. The method of claim 26, wherein the DNA of interest comprises tumor tissue DNA, transplant tissue DNA, or fetal tissue DNA.

28. The method of claim 1, wherein the method further comprises sequencing the plurality of cell-free linear DNA molecules to obtain the sequence reads.

29. The method of claim 1, wherein the biological sample comprises plasma.

30. The method of claim 1, wherein the plurality of cell-free linear DNA molecules comprises at least 1000 cell-free linear DNA molecules.

31. A computer product comprising a non-transitory computer readable medium storing a plurality of instructions that, when executed, cause a computer system to perform the method of claim 1.

32. A system comprising:
the computer product of claim 31; and
one or more processors for executing instructions stored on the computer readable medium.

33. A system comprising means for performing the method of claim 1.

34. A system comprising one or more processors configured to perform the method of claim 1.

35. A system comprising modules that respectively perform the steps of the method of claim 1.
